(19) United States
(12) Patent Application Publication
(10) Pub. No.: US 2015/0056171 A1
(43) Pub. Date: Feb. 26, 2015

(54) Methods of Predicting Clinical Outcome of Chronic Lymphocytic Leukemia

(71) Applicant: UNIVERSITY OF ROCHESTER, Rochester, NY (US)

(72) Inventors: Richard Burack, Rochester, NY (US); Jan Spence, Webster, NY (US)

(73) Assignee: University of Rochester, Rochester, NY (US)

(21) Appl. No.: 14/385,630

(22) PCT Filed: Mar. 15, 2013
(86) PCT No.: PCT/US13/31925

§ 371 Date: Sep. 16, 2014

Related U.S. Application Data
(60) Provisional application No. 61/611,856, filed on Mar. 16, 2012.

(51) Int. Cl. C12Q 1/68

(52) U.S. Cl. CPC C12Q 1/6886; C12Q 2600/118; C12Q 2600/156; C12Q 2600/16
USPC 424/93.7; 435/6.12; 435/6.11; 506/7

(57) ABSTRACT

Provided are methods of predicting the outcome of a chronic lymphocytic leukemia (CLL) in a subject. The methods comprise performing a polymerase chain reaction assay for an IGH locus on a nucleic acid from a biological sample from a subject with CLL, wherein the PCR assay amplifies a non-coding region of the IGH locus, sequencing a product from the PCR assay, and determining a level of mutation in the non-coding region of the IGH locus. An increased level of mutation in the non-coding region as compared to a control indicates a positive outcome for the subject with CLL. A decreased level or no mutation in the non-coding region as compared to a control indicates a poor outcome for the subject with CLL.

### RELATED APPLICATIONS

This application claims the benefit of U.S. Application No. 61/611,856, filed on Mar. 16, 2012 which is hereby incorporated herein in its entirety.

### BACKGROUND

Chronic lymphocytic leukemia is the most common lymphoid malignancy. The prognosis is highly variable and there are many parameters used to suggest prognosis. A critical parameter is the mutation status of the immunoglobulin gene, a feature that indicates the differentiation state of the leukemic cells.

### SUMMARY

Provided herein are methods of predicting the outcome of a chronic lymphocytic leukemia (CLL) in a subject using fast and reliable methods by evaluating the mutation status of non-coding regions in the IGH locus. The methods comprise performing a polymerase chain reaction (PCR) assay for an immunoglobulin heavy (IGH) locus on a nucleic acid from a biological sample from a subject with CLL, wherein the PCR assay amplifies a non-coding region of the IGH locus, sequencing a product from the PCR assay, and determining a level of mutation in the non-coding region of the IGH locus. An increased level of mutation in the non-coding region as compared to a control indicates a positive outcome for the subject with CLL. A decreased level of mutation as compared to the control indicates a poor outcome for the subject with CLL.

Also provided are kits for predicting the outcome of a CLL in a subject. The kit comprises a first primer and a second primer. The first primer and second primer are designed to perform a PCR assay for an IGH locus on a nucleic acid, wherein the PCR assay amplifies a non-coding region of the IGH locus.

Further provided are treatment methods of using the methods described above to determine the treatment of a subject with a CLL. The methods comprise performing a PCR assay for an IGH locus on a nucleic acid from a biological sample from a subject with CLL, wherein the PCR assay amplifies a non-coding region of the IGH locus, sequencing a product of the PCR assay, determining a low level of mutation or no mutation in the non-coding region as compared to a control, and providing a selected treatment (e.g., a stem cell transplant) to the subject.

The details of one or more embodiments are set forth in the accompanying drawings and the description below. Other features, objects, and advantages will be apparent from the description and drawings, and from the claims.

### DESCRIPTION OF DRAWINGS

FIG. 1 shows a diagram of the design of the multiplex PCR assay for amplification of the chronic lymphocytic leukemia (CLL)-immunoglobulin heavy (IGH) genomic DNA.

FIG. 2 shows an image of agarose gel electrophoresis analysis of Pool 1, Pool 3, and Pool 4 polymerase chain reaction (PCR) assays. The image shows a representative gel electrophoresis from 12 CLL patient specimens (specimen nos. 174-185) of the 3 multiplexed PCR reactions (Pool 1, Pool 3, and Pool 4). Each lane contains at most 1 DNA band and most patient specimens generated only 1 band within the 3 multiplexed PCR reactions. Sample 176* shows the typical result when both Chromosome 14 (Chr14) alleles are rearranged at the IGH loci, with amplicons generated with 2 different primer pools (Pools 3 and 4).

FIG. 3 shows a schematic of downstream J-intron insertions and deletions (indels). FIG. 3 shows that indels are common in B cells with mutated Vh families.

FIG. 4A shows a diagram of the Jh regions of IGH. FIG. 4B shows the sequence location and orientation of primers for sequencing Jh regions of IGH loci (SEQ ID NO:14).

FIG. 5 shows the sequence (SEQ ID NO:15) from the Jhl region through the downstream Pv259 (SEQ ID NO:13) primer near the Mu enhancer region for comparison of individual IGH sequences. The Jh coding sequences are denoted by named dotted arrows below their sequence. The entire region downstream of the junctional J of the IGH sequence is evaluated for the present of insertions and deletions (indels)>5, which indicate somatic hypermutation (SHM). If no indels are found, the 500 bases downstream from the junctional Jh region are evaluated for mutations. These regions are in upper case letters, and in cases where the regions overlap (J3-J4, J4-J5), the overlapped regions are in upper case letters and underlined. Mutations can be single base changes (1 each) and indels<6, with each indel counted as 1 event. The J % ID is calculated as [(500—no. of mutations)/500]×100.

FIG. 6 shows a graph demonstrating the size of the indel(s) present in the intronic-J region of CLL-IGH sequences.

FIG. 7 shows the alignment of Vh region specific primers aligned to functional Vh genes. FIG. 7A shows the alignment of Vh region 1 specific primers to functional Vh region 1 genes. FIG. 7B shows the alignment of Vh region 2 specific primers to functional Vh region 2 genes. FIG. 7C shows the alignment of Vh region 3 specific primers to functional Vh region 3 genes. FIG. 7D shows the alignment of Vh region 4 specific primers to functional Vh region 4 genes. FIG. 7E shows the alignment of Vh region 5 specific primers to functional Vh region 5 genes. FIG. 7F shows the alignment of Vh region 6 specific primers to functional Vh region 6 genes. FIG. 7G shows the alignment of Vh region 7 specific primers to functional Vh region 7 genes. DNA sequences were obtained and used to build working files from NCBI build 37.3. *(loc-m) refers to reference sequence location—number of mismatches between Vh gene reference sequence and designated primer. Sequences for each Vh gene listed in FIGS. 7A-G are provided and identified as SEQ ID NOs: 25-72. The sequences set forth in the alignments provided in FIGS. 7A-G are identified as SEQ ID NOs: 73-112.

FIG. 8 shows the relationship between the Vh and non-coding intron % identity, which shows that these contiguous regions of the same IGH molecule are subjected to the same biological process that generates mutations, resulting in highly correlated mutations rates.

FIG. 9 shows the distribution of mutations within intronic J-regions relative to V(D)J junction.

### DETAILED DESCRIPTION

The IGH loci is responsible for encoding immunoglobulins, proteins whose function is to bind foreign (non-self) molecules and eliminate them from the body. Thus, immunoglobulins play a critical role in body defenses against pathogens. Secreted immunoglobulins are also called antibodies and are produced by a subset of lymphocytes called B-cells or B-lymphocytes. The relatively small, specific chemical structures capable of binding antibodies are called antigens, and cells possess many surface features that can function as antigens.

To have the ability to bind to the wide and ever-changing surfaces of pathogens, a process evolved to generate diversity in the recognition and binding portions of immunoglobulins. This B-cell specific diversity is achieved in two ways. The first way is through the rearrangement of three genes within the immunoglobulin heavy (IGH) loci, to bring together genes from the Vh region (variable region), D region (diversity region) and Jh region (joining region). There are 51 functional Vh genes, divided into 7-families based on sequence similarities, 23 D genes and 6 Jh genes in humans, and, in conjunction with additional processes that occur during genomic rearrangement, these genes generate the diverse group of antigen binding sites of early B-cells. This V(D)J genomic recombination occurs in the bone marrow during B-cell development and generates virtually unique, surface-expressed immunoglobulins. Every B-cell needs to make a functional immunoglobulin and makes only one immunoglobulin through a process called allelic exclusion. This process allows V(D)J recombination to be complete on one allele, and only if the resulting gene is non-functional is the second allele allowed to recombine. Any given B-cell will only make only one functional immunoglobulin, and the probability of any newly formed B-cells producing identical immunoglobulin is extremely unlikely.

The second process by which immunoglobulin diversity is enhanced occurs once the B-cells have been released from the bone marrow into the circulation systems. When a B-cell encounters a specific antigen capable of interacting with its private immunoglobulin, a series of steps occur which increases the immunoglobulin binding efficiency for that antigen. This is a process of antibody affinity maturation, and it occurs in the germinal center of the lymph node and involves an enzyme called activation-induced cytidine deaminase (AID). This enzyme chemically modifies one of the nucleoside bases that make up DNA, generating mutations in the rearranged IGH loci that translates into differences in the immunoglobulin protein structure, altering the binding affinity for its specific antigen. Thus antigen affinity occurs through the process of somatic hypermutation (SHM) due to AID activity. B-cells that now make an affinity maturated immunoglobulin, defined as those that bind antigen faster and more tightly, survive and proliferate; while B-cells whose immunoglobulin bind antigen less efficiently die.

In B-cell cancers (leukemia and lymphoma), the unique immunoglobulin produced by the tumor cells are often used as molecular tags to identify the clonal B-cell population that gave rise to the disease. Studies of the immunoglobulins from chronic lymphocytic leukemia (CLL) have shown that this disease can be separated into 2 types based on the nature of the clonal immunoglobulin: those whose Vh gene has undergone SHM due to AID, and those whose Vh gene has not been changed, as assessed by DNA sequencing of the rearranged IGH molecule. Current analysis relies on the deviation of the Vh sequence from nominal germline sequence, which is determined as a % identity to a reference sequence available at NCBI or IMGT. A sequence identity for the Vh gene≧98% is deemed to be non-mutated, while those with <98% identity are considered to have undergone SHM.

The present methods relate to this phenomenon, but rely on detecting the type of CLL based on non-coding DNA regions adjacent to the coding IGH regions. The advantage of the present methods include, but is not limited to, the fact that evaluation of non-coding regions can lead to a more robust measure of AID activity, as there is no functional protein product being produced by the non-coding regions. This is in contrast to the evaluation of the Vh region coding region, which codes for a portion of a functional protein, and, thus, is less tolerant of mutations that result in significant changes in protein structure such as altered folding or premature truncation.

Provided herein are methods for predicting the outcome of a chronic lymphocytic leukemia (CLL) in a subject. The methods comprise performing a polymerase chain reaction (PCR) assay for an immunoglobulin heavy (IGH) locus on a nucleic acid from a biological sample from a subject with CLL, wherein the PCR assay amplifies a non-coding region of the IGH locus, sequencing a product from the PCR assay, and determining the presence or absence of mutation in the non-coding region of the IGH locus. The presence of mutations or an increased level of mutation in the non-coding region as compared to a control indicates a positive outcome for the subject with CLL. A decreased level of mutations or the absence of mutations as compared to the control indicates a poor outcome for the subject with CLL. Maintaining a sequence highly similar to a control can also indicate a poor outcome for the subject with CLL.

Predicting the outcome can, for example, mean predicting the time required for a first treatment (i.e., a first therapy) for a subject with CLL. By positive outcome, it is meant that the CLL is not aggressive and there will be a longer treatment free interval. By a poor outcome, it is meant that CLL is an aggressive CLL that requires treatment and is more likely to be lethal. Treatments for the subject with aggressive CLL can, for example, comprise multi-agent chemotherapy and/or stem cell transplants. Examples of multi-agent chemotherapy include, but are not limited to, fludarabine, cyclophosphamide, and rituximab (FCR); pentostatin, cyclophosphamide, and rituximab (PCR); fludarabine, cyclophosphamide, and mitoxantrone (FCM); cyclophosphamide, vincristine, and prednisone (CVP); and cyclophosphamide, doxorubicin, vincristine, and prednisone (CHOP).

Selecting a subject with CLL can, for example, comprise observing certain signs and symptoms including, but not limited to weakness, tiredness, weight loss, fever, night sweats, enlarged lymph nodes, anemia, shortage of white blood cells, and a shortage of platelets in a subject. To diagnose a subject with CLL, a physician will perform a physical exam and test blood samples bone marrow samples, lymph node samples, and/or spinal fluid to confirm the subject has CLL. Lab tests on the samples collected are known in the art and include, but are not limited to, complete blood count (CBC) test, microscopic exams (e.g., determination of size, shape, and other traits of a white blood cell), cytochemistry, flow cytometry, cytogenetics, and immunocytochemistry.

The IGH locus can, for example, comprise a Vh region, a diversity region, a joining region, and a downstream non-coding region that is between the V(D)J and C (constant) coding regions. The C region is a fourth coding region of the IGH molecule that does not get juxtaposed to the V(D)J coding region. The downstream non-coding region comprises a region downstream of the joining region. The non-coding region of the IGH locus can also be referred to as an intronic region. The Vh region, diversity region, and joining region comprise coding regions of the IGH locus. The present methods focus, for example, on the downstream non-coding region.

Optionally, the PCR assay amplifies both coding region and non-coding region of the IGH locus. The presence of mutations in the coding and non-coding regions as compared to a control indicates a positive outcome for the subject with CLL. The absence of mutation as compared to the control indicates a poor outcome for the subject with CLL.

As used herein, a control can be a sequence obtained from a subject without CLL or a sequence that has not undergone SHM from a subject with CLL. A control can also be an IGH reference sequence obtained from GenBank, for example, SEQ ID NO: 15. SEQ ID NO: 15 is an example of a genomic IGH sequence that allows identification of mutations in coding and noncoding regions (intronic J regions) of an IGH sequence. One of skill in the art can use BLAST to compare the reference sequence to the IGH sequence from a subject. Using this technique, one of skill in the art can routinely compare two sequences and obtain the percentage identity (ID) between the sequences in order to determine if SHM has occurred. For example, a sample that is <98% ID, indicates that SHM has occurred in the subject. One of skill in the art can compare the sequence of the subject with more than one IGH sequence observed in the germline of subjects without CLL or an IGH sequence observed in a subject with CLL that has not undergone SHM in order to identify mutations, or lack therof, in the IGH sequence of a subject.

In another example, one of skill in the art can compare the IGH sequence from a subject with a database of reference IGH sequences available from the Immunogenetics Information System by using V-Quest. V-quest is readily available from the Immunogenetics Information System (http://www.imgt.org/IMGT_vquest/vquest?) (See Brochet, X. et al., Nucl. Acids Res. 36, W503-508 (2008). One of skill in the art can also compare the IGH sequence from a subject with a database of reference IGH sequences readily available from the National Center for Biotechnology (NCBI) htt://www.ncbi.nlm.nih.gov/igblast/). Both V-quest and IgBLAST allow analysis of mutations in the coding regions of an IGH sequence.

The PCR assay can, for example, be performed with a first primer and a second primer. Optionally, the first primer hybridizes with the Vh region or a portion thereof The Vh region can comprise a translational start site. Optionally, the first primer hybridizes with the Vh region at or near the translational start site. Hybridizing at the translational start site means that at least a portion of the primer hybridizes with at least a portion of the translational start site. The primer can, for example, completely hybridize with the translational start site. Hybridizing near the translational start site means that the primer hybridizes between 1 to 150 bases of the translational start site. To limit the effects of mutation on primer binding sites, Vh region primers are designed to hybridize as close to the promoter as possible.

Optionally, the Vh region is selected from the group consisting of Vh region 1, Vh region 2, Vh region 3, Vh region 4, Vh region 5, Vh region 6, and Vh region 7. By Vh region 1, Vh region 2, Vh region 3, Vh region 4, Vh region 5, Vh region 6, and Vh region 7, it is meant to include all Vh genes in the Vh1 family, Vh2 family, Vh3 family, Vh4 family, Vh5 family, Vh6 family, and Vh7 family, respectively. The various Vh genes arose thorough gene duplication and are grouped into families based on sequence homology. The gene families are not contiguous on the chromosome, but are often highly interspersed. Vh genes and families are known in the art. See, e.g., Tobin, Ups. J. Med. Sci. 110(2):97-113 (2005); Chowdhury and Sen, Immunol. Rev. 200:182-96 (2004).

Optionally, the first primer is selected from the group consisting of SEQ ID NOs:1-12. Optionally, the Vh region 1 hybridizing primer comprises SEQ ID NO:1 or SEQ ID NO:2. Optionally, the Vh region 2 hybridizing primer comprises SEQ ID NO:8. Optionally, the Vh region 3 hybridizing primer comprises SEQ ID NO:5, SEQ ID NO:6, or SEQ ID NO:7. Optionally, the Vh region 4 hybridizing primer comprises SEQ ID NO:9, SEQ ID NO:10, or SEQ ID NO:11. Optionally, the Vh region 5 hybridizing primer comprises SEQ ID NO:3. Optionally, the Vh region 6 hybridizing primer comprises SEQ ID NO:12. Optionally, the Vh region 7 hybridizing primer comprises SEQ ID NO:4.

Optionally, the second primer hybridizes with a non-coding region downstream of the joining region. The non-coding region downstream of the joining region can, for example, be unaffected by activation-induced cytidine deaminase (AID) activity. To limit the effects of mutation on the primer binding sites, the second primer is designed to be beyond the AID activity window. The second primer can, for example, hybridize with the non-coding region about 800 bases to about 3000 bases downstream of the V(D)J junction depending on which J region is used in the V(D)J junction. Optionally, the second primer hybridizes about 1000 bases downstream of the junction region. By about, it is meant that the primer can hybridize between 0 and 100 bases of the range or number provided (i.e., 900-3000 bases). Optionally, the second primer comprises SEQ ID NO:13.

Optionally, the PCR assay comprises at least two first primers and the second primer, wherein the primers are in a primer pool. By primer pool, it is meant that there are more than two primers for the PCR reaction in one solution and there at least two potential PCR products. By way of an example, a primer pool can comprise a Vh region 1, Vh region 2, and downstream non-coding hybridizing primer in a single PCR reaction solution, which will produce a PCR product comprising either Vh region 1 or Vh region 2 depending on which Vh gene was included in the V(D)J recombination event. Optionally, the at least two first primers hybridize with a Vh region or a portion thereof. Optionally the Vh region comprises a translation start site. The at least two primers can, for example, hybridize at or near the translation start site. Hybridizing at the translation start site means that at least a portion of the primer hybridizes with at least a portion of the translation start site. The primer can, for example, completely hybridize with the translation start site. Hybridizing near the translation start site means that the primer hybridizes between 1 to 150 bases of the translation start site.

Optionally, the Vh region is selected from the group consisting of Vh region 1, Vh region 2, Vh region 3, Vh region 4, Vh region 5, Vh region 6, and Vh region 7. Optionally, the at least two first primers comprise two or more primers selected from the group consisting of SEQ ID NOs:1-12. Optionally, the primer pool comprises a Vh region 1 hybridizing primer and at least one of a Vh region 2, 3, 4, 5, 6, or 7 hybridizing primer. Optionally, the primer pool comprises a Vh region 2 hybridizing primer and at least one of a Vh region 3, 4, 5, 6, or 7 hybridizing primer. Optionally, the primer pool comprises a Vh region 3 hybridizing primer and at least one of a Vh region 4, 5, 6, or 7 hybridizing primer. Optionally, the primer pool comprises a Vh region 4 hybridizing primer and at least one of a Vh region 5, 6, or 7 hybridizing primer. Optionally, the primer pool comprises a Vh region 5 hybridizing primer and at least one of a Vh region 6 or 7 hybridizing primer. Optionally, the primer pool comprises a Vh region 6 hybridizing primer and a Vh region 7 hybridizing primer.

Optionally, the primer pool comprises a Vh region 1 hybridizing primer, a Vh region 5 hybridizing primer, and a Vh region 7 hybridizing primer. The Vh region 1 hybridizing primer can, for example, comprise SEQ ID NO:1 or SEQ ID NO:2, the Vh region 5 hybridizing primer can, for example, comprise SEQ ID NO:3, and the Vh region 7 hybridizing primer can, for example, comprise SEQ ID NO:4. Optionally, the primer pool comprises a Vh region 3 hybridizing primer and a Vh region 2 hybridizing primer. The Vh region 3 hybridizing primer can, for example, comprise SEQ ID NO:5, SEQ ID NO:6, or SEQ ID NO:7, and the Vh region 2 hybridizing primer can, for example, comprise SEQ ID NO:8. Optionally, the primer pool comprises a Vh region 4 hybridizing primer and a Vh region 6 hybridizing primer. The Vh region 4 hybridizing primer can, for example, comprise SEQ ID NO:9, SEQ ID NO:10, or SEQ ID NO:11, and the Vh region 6 hybridizing primer can, for example, comprise SEQ ID NO:12.

The second primer of the primer pool can, for example, hybridize with a non-coding region downstream of the junctional region as described above.

Optionally, the nucleic acid of the sample is DNA. Optionally, ribonucleic acid (RNA) can be used instead of DNA in any of the methods or compositions described herein. For example, RNA comprising the transcribed IGH locus is isolated from a biological sample from the subject. The RNA is reverse transcribed and PCR amplified to prepare cDNA, which can be used in the methods described herein. Reverse transcription and PCR amplification techniques are known in the art. See, e.g., Ausubel et al., Current Protocols in Molecular Biology (John Wiley & Sons, Inc., New York (1999); and Sambrook et al., Molecular Cloning: A Laboratory Manual, 3^{rd} Ed., Cold Spring Harbor Press, Cold Spring Harbor, N.Y. (2001).

Optionally, the biological sample from the subject is a clinical sample. A biological sample can be, without limitation, a cellular sample, a tissue sample, a diagnostic biopsy sample, or a fluid sample. For example, cells include, without limitation, peripheral blood mononuclear cells (PBMCs), leukocytes, tissue explants, or cells lines derived from the subject. Diagnostic biopsy samples include, for example, lymph node biopsies, tonsil biopsies, bone marrow biopsies, or any biopsy of healthy or diseased tissue. Biological fluid samples include, for example, a blood sample, a lymph sample, a plasma sample, a urine sample, a sputum sample, a saliva sample, or a cerebrospinal fluid sample. Biological samples can be collected from an individual using any standard method known in the art that results in the preservation of nucleic acids. Blood samples can be obtained via venous puncture techniques. Serum samples can be prepared from whole blood using standard methods such as centrifuging blood samples that have been allowed to clot. Plasma samples can be obtained by centrifuging blood samples that were treated with an anti-coagulant such as heparin. Saliva samples may be collected using cotton swabs, wipes, suction, or scraping. Biopsies can be collected using standard techniques such as needle biopsy or surgical excision.

Sequencing of the PCR product can, for example, comprise using the Sanger Method or any method known to the artisan. Optionally, the PCR product comprises a bar code or tag. The bar code or tag can, for example, be used to sequence the PCR product using a deep sequencing method. Sequencing methods, including deep sequencing methods, are known in the art. See, e.g., Lee and Tang, Methods Mol. Biol. 855:155-74 (2012); Shendure et al., Curr. Protoc. Mol. Biol., Chapter 7:Unit 7 (2011); Bao et al., J. Hum. Genet. 56(6):406-14 (2011); Fox et al., Methods Mol. Biol. 553:79-108 (2009); Ausubel et al., Current Protocols in Molecular Biology (John Wiley & Sons, Inc., New York (1999); and Sambrook et al., Molecular Cloning: A Laboratory Manual, 3^{rd} Ed., Cold Spring Harbor Press, Cold Spring Harbor, N.Y. (2001)

Also provided are kits for predicting the outcome of a chronic lymphocytic leukemia (CLL) in a subject. The kits comprise a first primer and a second primer. The first primer and second primer are for performing a PCR assay for an IGH locus on the nucleic acid, wherein the PCR assay amplifies a non-coding region of the IGH locus.

The first primer of the kit is selected from the first primers described above. Briefly, the first primer optionally, hybridizes with the Vh region, for example. Optionally, the Vh region is selected from the group consisting of Vh region 1, Vh region 2, Vh region 3, Vh region 4, Vh region 5, Vh region 6, and Vh region 7. Optionally, the first primer is selected from the group consisting of SEQ ID NOs:1-12. A kit can comprise one or more first primers and optionally include any combination of primers directed to Vh region 1, Vh region 2, Vh region 3, Vh region 4, Vh region 5, Vh region 6, or Vh region 7.

Optionally, the second primer of the kit hybridizes with a non-coding region downstream of the joining region as described above. Optionally, the kit comprises more than one second primer and the user can select from among the second primers for use in the assay.

Optionally, the kit comprises at least two first primers and the second primer, wherein the primers are in a primer pool. The two first primers are different from each other and optionally are directed to different Vh regions (e.g., any combination of Vh region 1, Vh region 2, Vh region 3, Vh region 4, Vh region 5, Vh region 6, and Vh region 7). The second primer is as described above. Optionally, the kit includes more than two first primers and the user can select from among the assortment to include in the primer pool.

Optionally, the kit comprises containers for the primers, vessels for the PCR reactions to occur, buffers for the PCR reactions, nucleotides for the PCR reaction, and one or more polymerases for the PCR reaction. PCR containers, buffers, nucleotides, and polymerases are known in the art. See, for example, Ausubel et al., Short Protocols in Molecular Biology, 5th ed., Wiley & Sons, 2002 and Sambrook et al., Molecular Cloning: A Laboratory Manual, 3^{rd} ed., Cold Spring Harbor, N.Y., 2001).

Also provided are methods of treating a subject with a chronic lymphocytic leukemia (CLL) based on the results of the assay as described herein. The methods, as described above, comprise performing a PCR assay for an IGH locus on a nucleic acid from a biological sample from the subject with CLL wherein the PCR assay amplifies a non-coding region of the IGH locus, sequencing a product from the PCR assay, determining the presence or absence of a mutation in the non-coding region as compared to a control. The treatment method further comprises providing a selected treatment (e.g., a multi-agent chemotherapy or stem cell transplant) to the subject. As used herein, a control can be a sequence obtained from a subject without CLL or a sequence that has not undergone SHM from a subject with CLL. A control can also be a reference sequence obtained from immunoglobulin-blast (IgBLAST) from the National Center for Biotechnology Information. Optionally, the control sequence is SEQ ID NO:15.

Stem cell transplants can, for example, comprise providing stem cells to the subject from the same subject (prior to CLL or healthy stem cells from the same subject) or from a different subject. The stem cells can be embryonic stem cells or adult stem cells. Stem cell therapies are known in the art. See, e.g., Freed et al., Bone Marrow Transplant (Dec. 2011); Titomanilo et al., Ann. Neurol. 70(5):698-712 (2011); Alfaro et al., Vitam. Horm. 87:39-59 (2011); Choudry and Mathur, Regen Med. 6(6 Suppl):17-23 (2011); and Lunn et al., Ann. Neurol. 70(3):353-61 (2011).

As described herein, any method of polymerase chain reaction (PCR) can be used as long as it generates products that are suitable for sequencing. To limit amplification of off-target products, all primer are designed to function with stringent PCR conditions, with most primers having a Tm>65° C. PCRs are performed by standard methods. See, for example, Ausubel et al., Short Protocols in Molecular Biology, 5th ed., Wiley & Sons, 2002 and Sambrook et al., Molecular Cloning: A Laboratory Manual, 3^{rd} ed., Cold Spring Harbor, N.Y., 2001) or using commercially available reagents or kits. Representative suppliers of such reagents or kits include Invitrogen (Carlsbad, Calif.), Stratagene (La Jolla, Calif.), Agilent Technologies (Santa Clara, Calif.) and Affymetrix (Santa Clara, Calif.). Reaction conditions will vary according to a number of factors, including, for example, the primer and target DNA sequence, the length of the products desired, the nature of the label, and the specific DNA polymerase that is used. Useful DNA polymerases include, for example, Taq DNA polymerase, modified Taq DNA polymerases or other DNA polymerases in which the 3′ exonuclease activity has been attenuated or eliminated relative to that of the wild type polymerase, e.g., exo-Pfu DNA polymerase or exo-Klenow fragment.

As used throughout, subject can be a vertebrate, more specifically a mammal (e.g., a human, horse, cat, dog, cow, pig, sheep, goat, mouse, rabbit, rat, and guinea pig), birds, reptiles, amphibians, fish, and any other animal. The term does not denote a particular age or sex. Thus, adult and newborn subjects, whether male or female, are intended to be covered. As used herein, patient or subject may be used interchangeably and can refer to a subject with a disease or disorder (e.g., CLL). The term patient or subject includes human and veterinary subjects.

According to the methods taught herein, the subject is administered an effective amount of stem cells or chemotherapeutic agents. The term effective amount is defined as any amount necessary to produce a desired physiologic response. Effective amounts and schedules for administering the stem cells or chemotherapeutic agents may be determined empirically, and making such determinations is within the skill in the art. Chemotherapeutic agents can be delivered via numerous routes, including, but not limited to, oral, intravenous or subcutaneous administration. Stem cells can be delivered via injection or infusion, for example. The ranges for administration are those large enough to produce the desired effect (e.g., treating the subject with CLL). The dosage should not be so large as to cause substantial adverse side effects, such as unwanted cross-reactions, anaphylactic reactions, and the like. Generally, the amount will vary with the age, condition, sex, etc. can be determined by one of skill in the art.

As used herein the terms treatment, treat, or treating refers to a method of reducing the effects of a disease or condition or symptom of the disease or condition. Thus in the disclosed method, treatment can refer to a 10%, 20%, 30%, 40%, 50%, 60%, 70%, 80%, 90%, or 100% reduction in the severity of an established disease or condition or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus , if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### EXAMPLES

The IGH loci requires chromosomal rearrangement to juxtapose Vh, D and Jh segments to make a functional immunoglobulin gene. Additional immunoglobulin diversification can be generated by somatic hypermutation (SHM). CLL is a clonal proliferation of B-cells with 2 clinical patterns that differ in aggressiveness, best predicted by Vh mutation status.

Materials and Methods

Patient Selection. Peripheral blood samples from patients diagnosed with chronic lymphocytic leukemia (CLL) were obtained from the Hematopoietic Malignancy Tissue Procurement Core of Strong Memorial Hospital, Rochester NY. These samples were stored at in liquid nitrogen with cryopreservatives or at −80° C. as simple blood pellets. Specimens were anonymized through a tissue procurement protocol approved by the University of Rochester Research Subjects Review Board; the protocol allows blinded access to clinical information and limited patient health information (PHI).

DNA extraction. DNA was extracted with QIAamp DNA mini Kit (Qiagen Inc., Valencia, Calif.) for samples with >4×10⁻⁶ white blood cells or Wizard Genomic DNA purification Kit (Promega Corp, Madison Wis.) for samples containing between 2 and 4×10⁻⁶ white blood cells. DNA concentration was estimated by spectrophotometry using the Nanodrop ND-1000 (Wilmington, Del.).

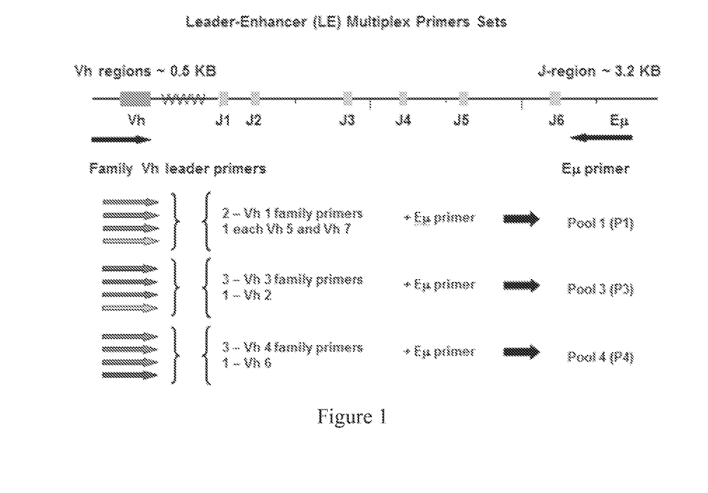

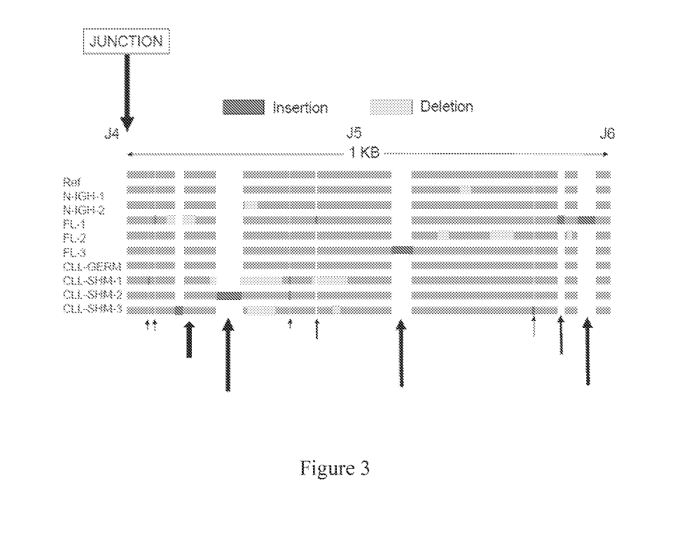

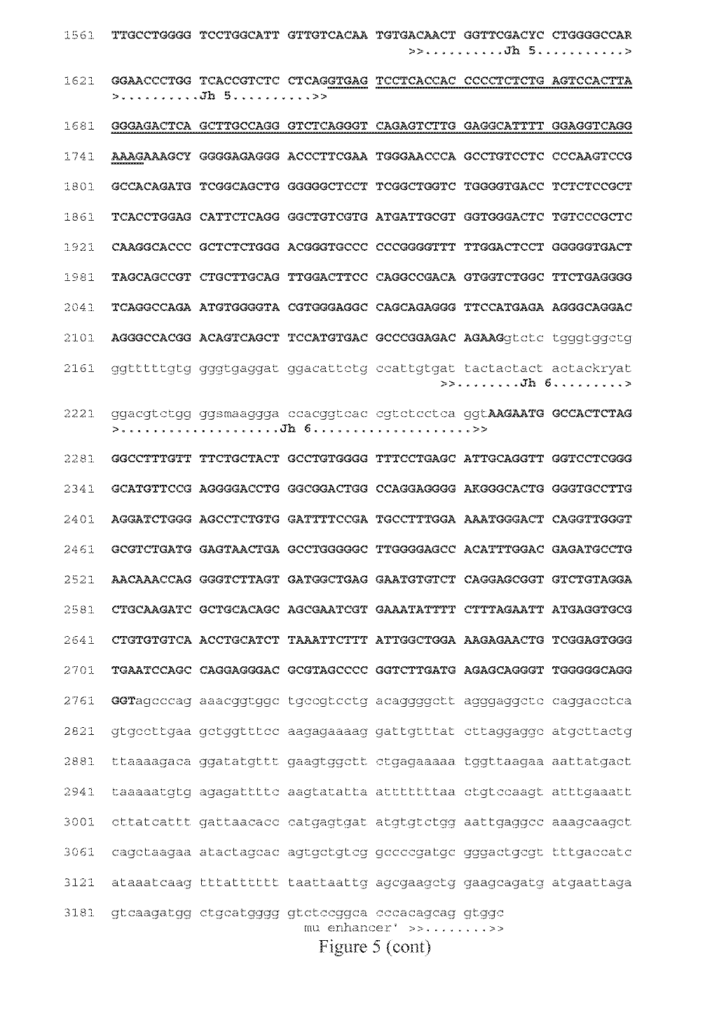

PCR amplification. For amplification of clonal IGH in patient specimens, patient tumor DNA (approximately 150 ng per 50 μl reaction) was amplified using HotStarTaq Plus (Qiagen, Inc) with CoralLoad PCR buffer according to manufacturer's recommendations. Three sets of multiplex PCR reactions were performed on each patient sample (FIG. 1, Table 1): Pool 1 contained primers Pv259 (SEQ ID NO:13), Pv367 (SEQ ID NO:1), Pv385 (SEQ ID NO:2), Pv 378 (SEQ ID NO:3) and Pv375 (SEQ ID NO:4); Pool 3 contained primer Pv259 (SEQ ID NO:13), Pv383 (SEQ ID NO:5), Pv382 (SEQ ID NO:6), Pv 374 (SEQ ID NO:7) and Pv384 (SEQ ID NO:8); Pool 4 contained primers Pv259 (SEQ ID NO:13), Pv380 (SEQ ID NO:9), Pv381 (SEQ ID NO:10), Pv 379 (SEQ ID NO:11) and Pv376 (SEQ ID NO:12). All primers were used at 0.2 μM each. Thermocycler (BioRad MyCycler, Hercules Calif.) settings were 95° C. for 5 minutes followed by 35 cycles of 94° C. for 45 seconds, 63° C. for 30 sec, to 72° C. for 4 minutes. Products were completed with 10 minutes at 72° C. PCR reactions were analyzed on a 0.7% agarose gel containing 1% ethidium bromide (Sigma-Aldrich, St. Louis Mo.) and visualized with UV-transillumination. Amplicons were purified with QIAquick PCR purification spin columns (Qiagen, Inc), and DNA concentration was estimated by spectrophotometry using the Nanodrop ND-1000 and samples sequenced by Genewiz, Inc (South Plainfield, N.J.). IGH sequence results, from duplicated sequencing runs, were analyzed for Vh, D and J usage and mutation status using IgBLAST and confirmed at IMGT/V-QUEST. Intronic region alignments were generated with Clone Master (Scientific & Educational Software, Cary, N.C.) against IGH reference sequence obtained from NCBI Build 37.3 (accessed 03.09.2012). This extended J-reference table (FIG. 5) contains the sequence (SEQ ID NO:15) from Jh1 through the downstream Pv259 (SEQ ID NO:13) primer near the Mu enhancer region for comparison of individual IGH sequence. The entire region downstream of the junctional J of the IGH sequence is evaluated for the presence of insertions and deletions (indels)>5 bases (FIG. 3), which indicate somatic hypermutation (SHM). If no indels>5 bases are found, the 500 bases downstream from the junctional Jh region are evaluated for mutations. These various J regions are in bold, and in cases where the regions overlap, the overlapped regions are bold and underlined. Mutations include single base changes (1 each) and indels<6 bases, with each indel counted as 1 event, analogous to methodology used for Vh % identity. The J % ID is calculated as (500−number of mutations)/500.

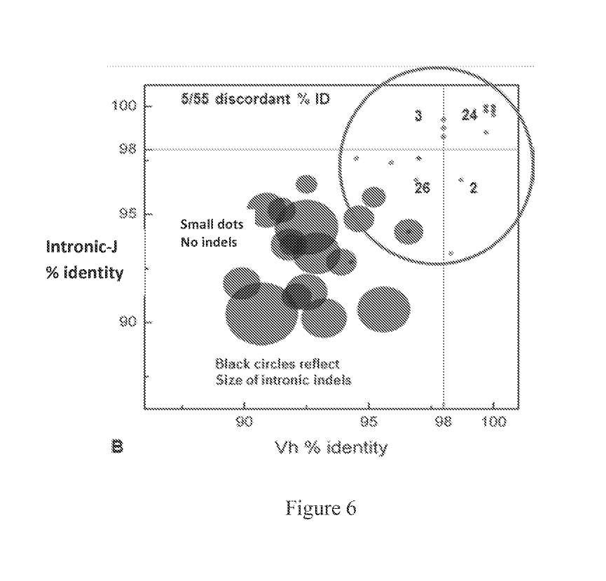

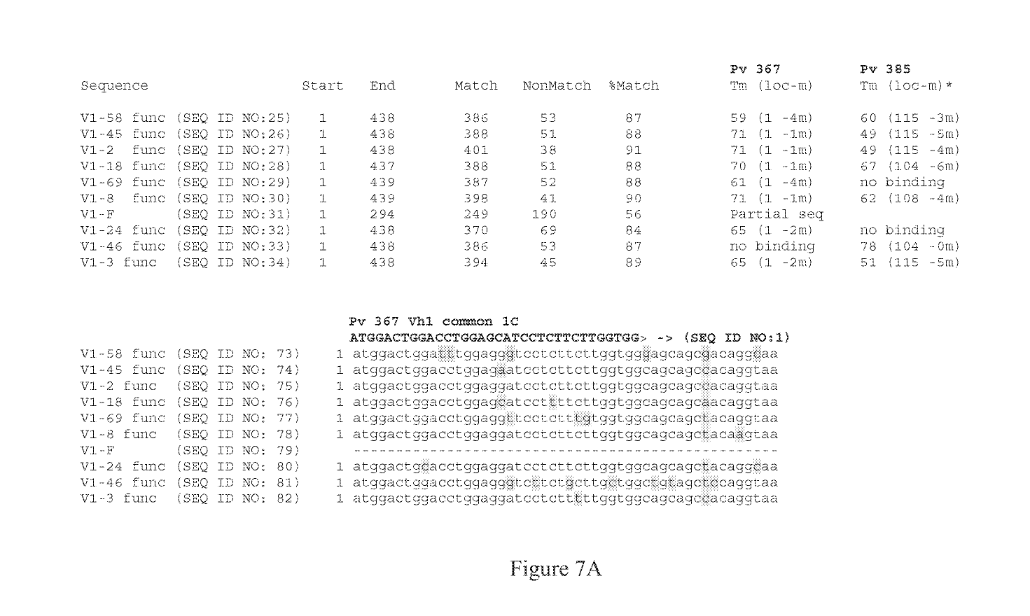

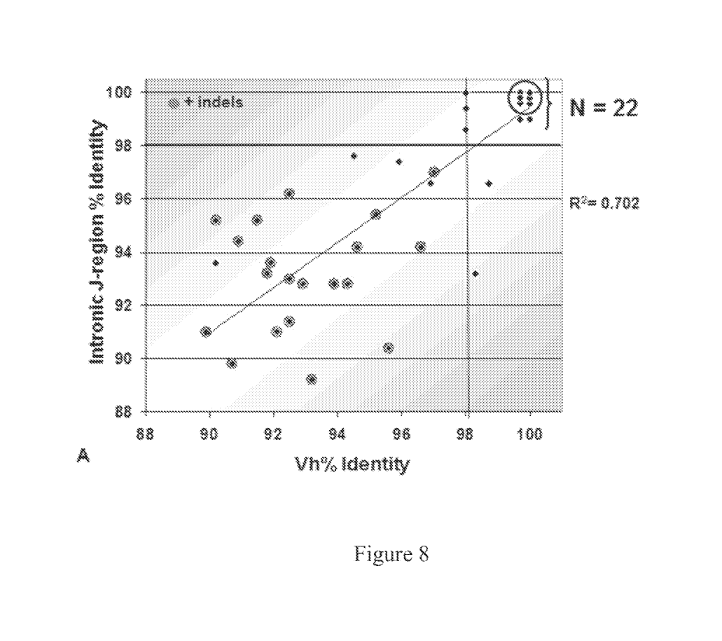

The size of the indel(s) present in the intronic-J region of CLL-IGH sequences (FIG. 6) and the correlation between Vh and intronic J region mutation rates (FIG. 8) was also determined. The linear correlation between Vh and intronic J region mutation rates is consistent with both mutations rates arising from the same biological process.

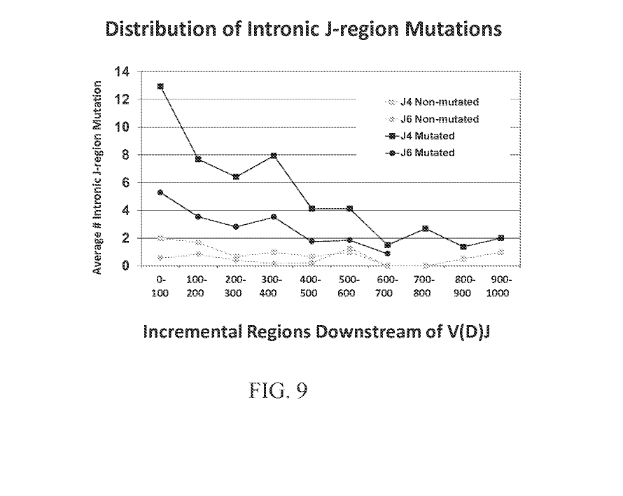

The distribution of mutations within the Intronic J-region relative to V(D)J junction was also determined (FIG. 9). Mutations counted in accordance with Vh % ID standards, were averaged over 100 base-pair increments starting from the IGH junctions from IGH using either J4 (squares) or J6 (circles), the overwhelmingly predominant Jh genes used in V(D)J junctions in IGH from CLL (see Table 3).

TABLE 1
Primers for multiplex PCR for amplification of CLL-IGH
genomic DNA.

| | | | | Tm |
|---|---|---|---|---|
| Pool | site | ID# | Sequence (5′-3′) | ° C. |
| 1, 3 & 4 | Eμ | Pv259 | GCCACCTGCTGTGGGTGCCGGAGAC | 77 |
| | | | (SEQ ID NO: 13) | |
| 1 | Vh1 | Pv367 | ATGGACTGGACCTGGAGCATCCTCTTCTTGGTGG | 76 |
| | | | (SEQ ID NO: 1) | |
| 1 | Vh1 | Pv385 | GTCATTCTCTACTGTGTCCTCTCCGCAGGTGCTCACTCCC | 78 |
| | | | (SEQ ID NO: 2) | |
| 1 | Vh5 | Pv378 | CCTCGCCCTCCTCCTGGCTGTTCTCC | 75 |
| | | | (SEQ ID NO: 3) | |
| 1 | Vh7 | Pv375 | CTTCTTGATGGCAGCAGCAACAGGTAAGG | 71 |
| | | | (SEQ ID NO: 4) | |
| 3 | Vh3 | Pv383 | ATGGAGTTGGGGCTGAGCTGGGTTTTCC | 74 |
| | | | (SEQ ID NO: 5) | |
| 3 | Vh3 | Pv382 | GAAACAGTGGATACGTGTGGCAGTTTCTGAC | 70 |
| | | | (SEQ ID NO: 6) | |
| 3 | Vh3 | Pv374 | GAAACAGTGGATTTGTGTGGCAGTTTCTGAC | 70 |
| | | | (SEQ ID NO: 7) | |
| 3 | Vh2 | Pv384 | TTGTCTCCTTTGTGGGCTTCATCTTCTTATG | 68 |
| | | | (SEQ ID NO: 8) | |
| 4 | Vh4 | Pv380 | ATGAAACACCTGTGGTTCTTCCTCCTGCTG | 71 |
| | | | (SEQ ID NO: 9) | |
| 4 | Vh4 | Pv381 | ATGAAACACCTGTGGTTCTTCCTCCTCCTG | 71 |
| | | | (SEQ ID NO: 10) | |
| 4 | Vh4 | Pv379 | CTGGTGGCAGCTCCCAGATGTGAGTATCTC | 72 |
| | | | (SEQ ID NO: 11) | |
| 4 | Vh6 | Pv376 | ATGTCTGTCTCCTTCCTCATCTTCCTGC | 69 |
| | | | (SEQ ID NO: 12) | |

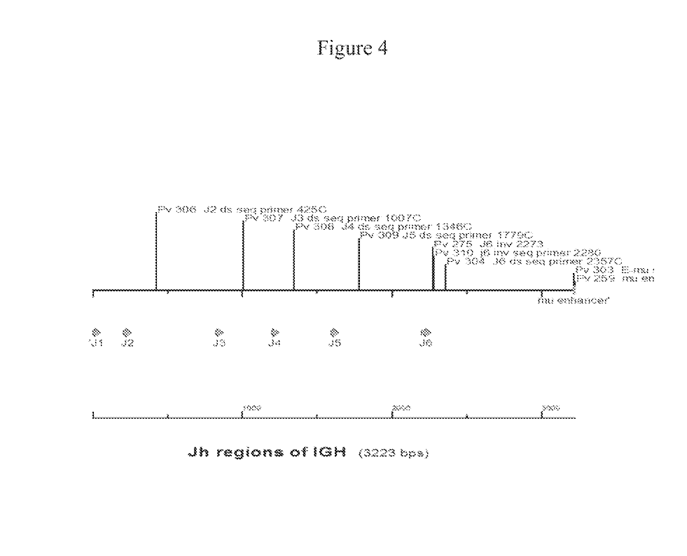

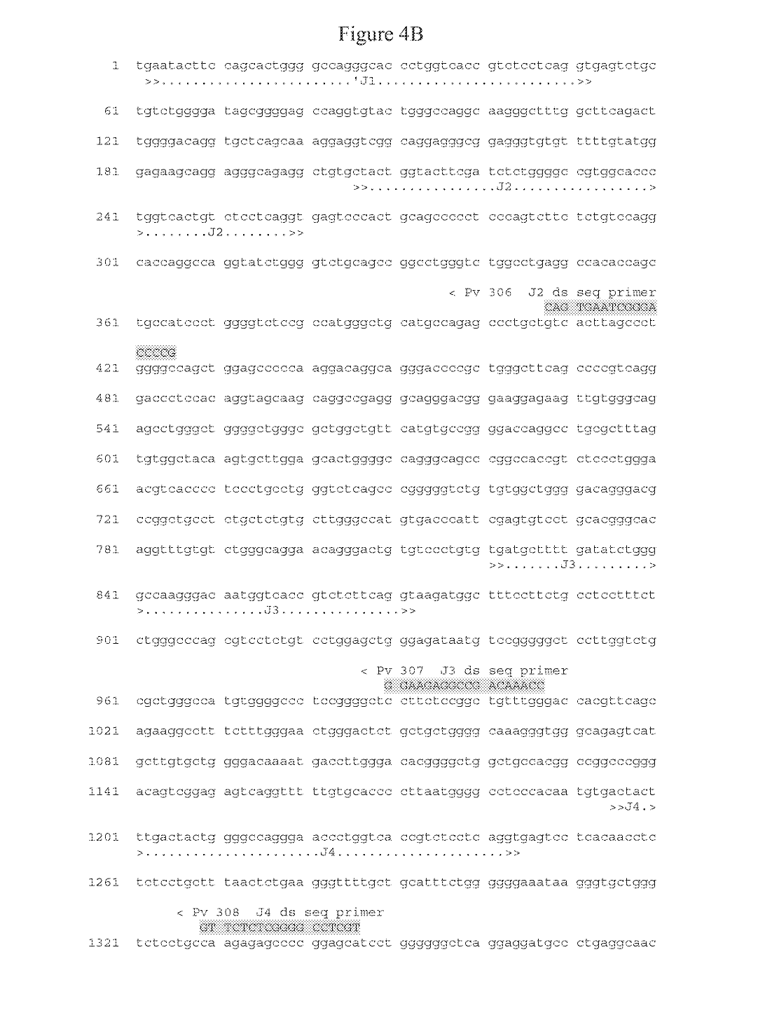

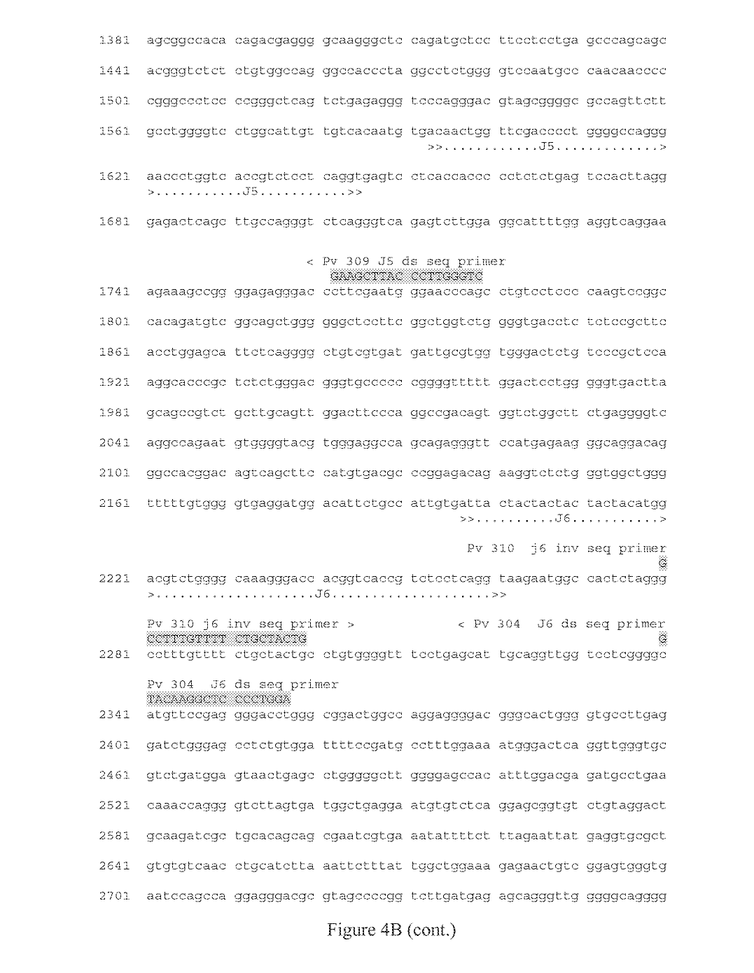

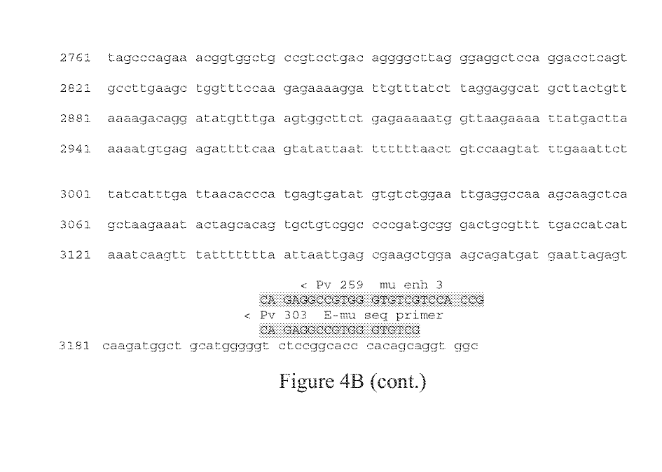

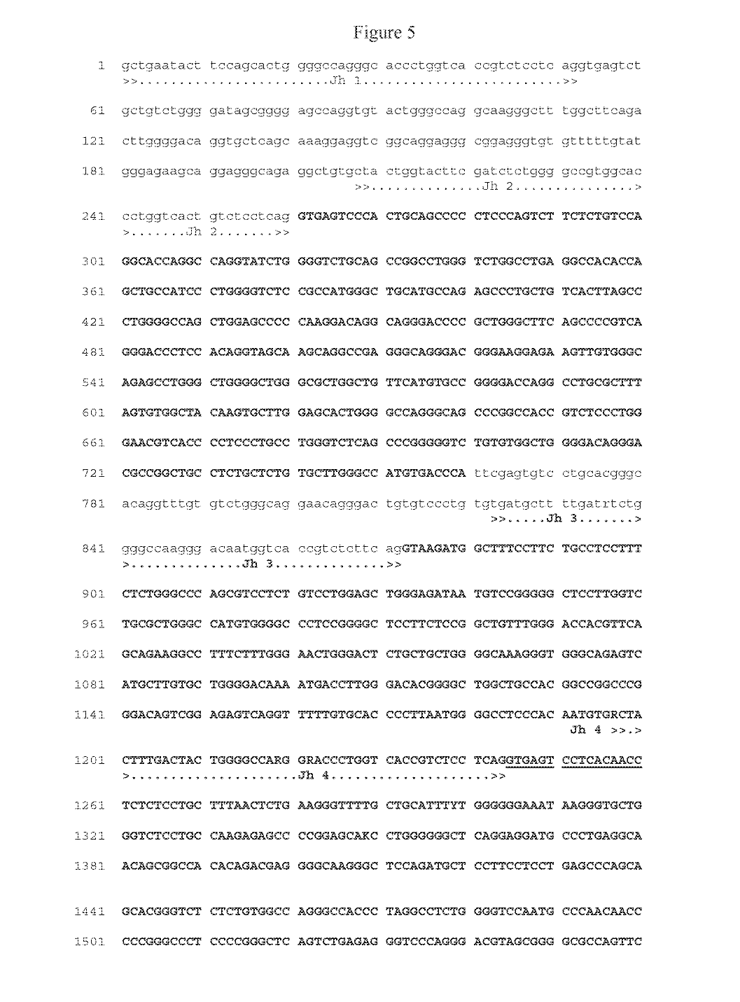

TABLE 2
Primers used for sequencing of IGH amplicons. See sequence
location of FIG. 4B for location and orientation of listed
primers.

| | | | | Tm |
|---|---|---|---|---|
| Primer | region | sequence (5′-3′) | purpose | (° C.) |
| Pv 259 | Eμ | GCCACCTGCTGTGGGTGCCGGAGAC | IGH | 77 |
| | | (SEQ ID NO: 13) | amplification | |
| Pv 303 | Eμ | GCTGTGGGTGCCGGAGAC | sequencing | 67 |
| | | (SEQ ID NO: 16) | | |
| Pv 235 | J6 | CGCCCAGGTCCCCTCGGAACATGCC | IGH | 76 |
| | | (SEQ ID NO: 17) | amplification | |
| Pv 304 | J6 | AGGTCCCCTCGGAACATG | sequencing | 62 |
| | | (SEQ ID NO: 18) | | |
| Pv 310 | J6 | GCCTTTGTTTTCTGCTACTG | sequencing | 59 |
| | | (SEQ ID NO: 19) | | |
| Pv 309 | J5 | CTGGGTTCCCATTCGAAG | sequencing | 59 |
| | | (SEQ ID NO: 20) | | |
| Pv 308 | J4 | TGCTCCGGGGCTCTCTTG | sequencing | 65 |
| | | (SEQ ID NO: 21) | | |
| Pv 307 | J3 | CCAAACAGCCGGAGAAGG | sequencing | 62 |
| | | (SEQ ID NO: 22) | | |
| Pv 306 | J2 | GCCCCAGGGCTAAGTGAC | sequencing | 64 |
| | | (SEQ ID NO: 23) | | |
| Pv 305 | J1 | CTGAAGCCAAAGCCCTTG | sequencing | 60 |
| | | (SEQ ID NO: 24) | | |

TABLE 3
Distribution of junctional
J usage and presence of Indels based on Vh % ID.

| Vh % ID | Jh-2 | Jh-3 | Jh-4 | Jh-5 | Jh-6 |
|---|---|---|---|---|---|
| ≧98%: J use | 1 | 0 | 7 | 0 | 19 § |
| <98%: J use | 1 | 3 | 18 § | 2 | 4 |
| ≧98%: +Indel * | 0 | 0 | 0 | 0 | 0 |
| <98%: +indel * | 1 | 1 | 17 | 9 | 2 |

§ The junctional J-usage pattern is statistically different (p < 0.0001) by Fisher exact test.
* The distribution of indels is statistically different (p < 0.0001) by Fisher exact test.

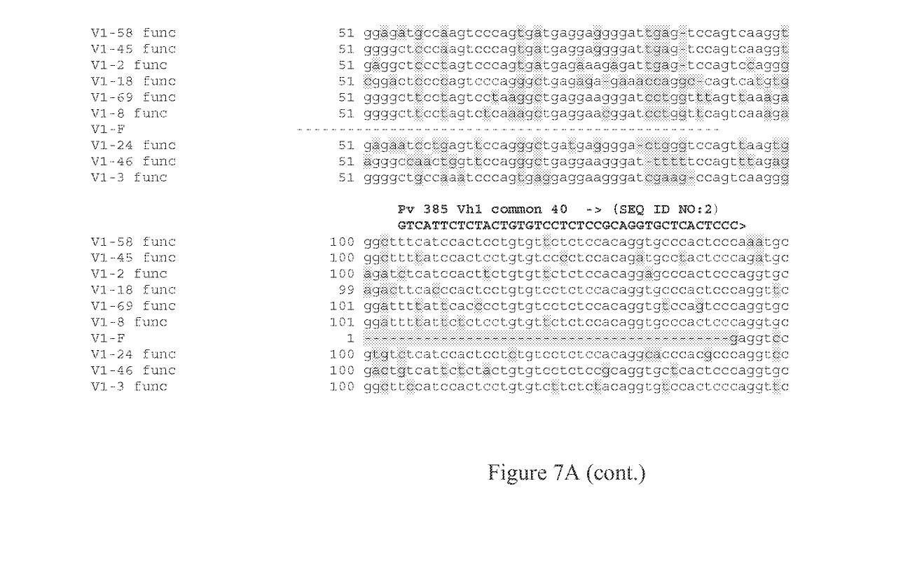

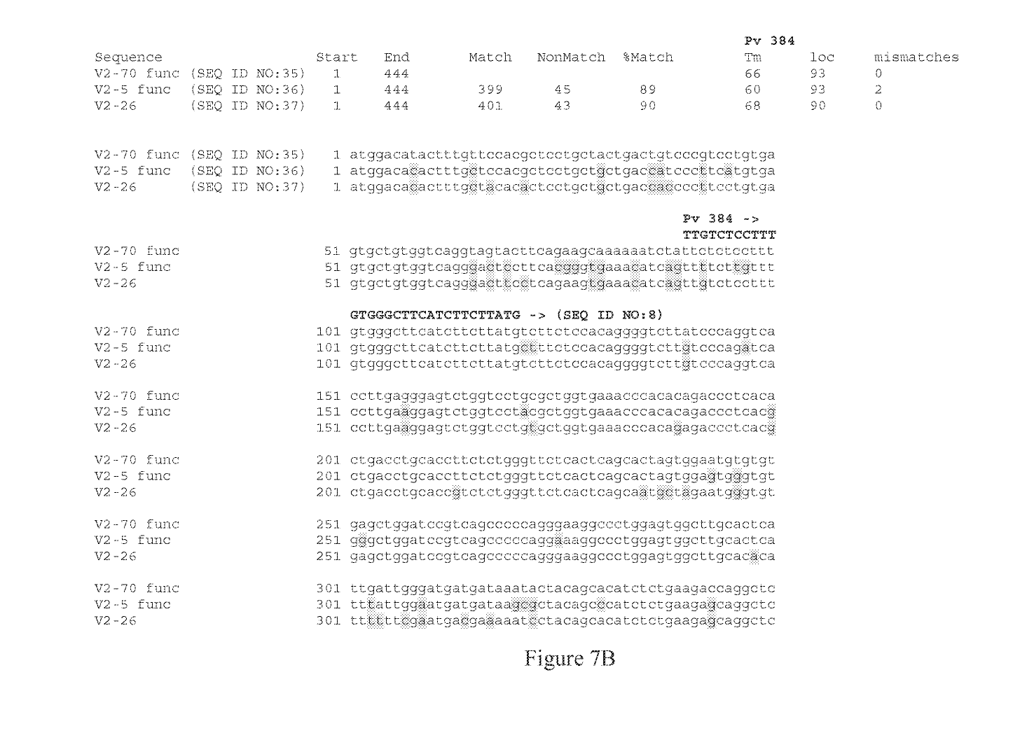

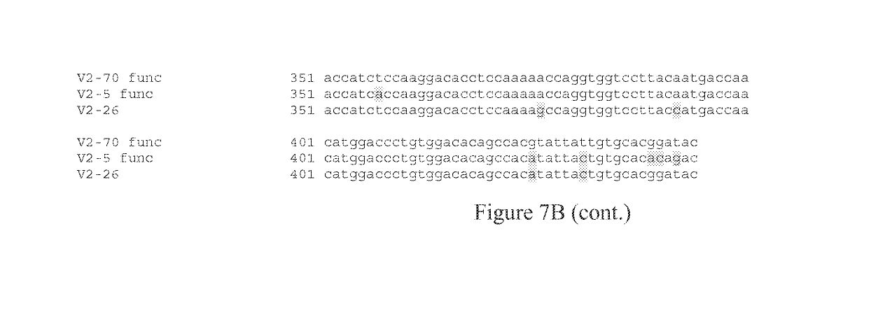

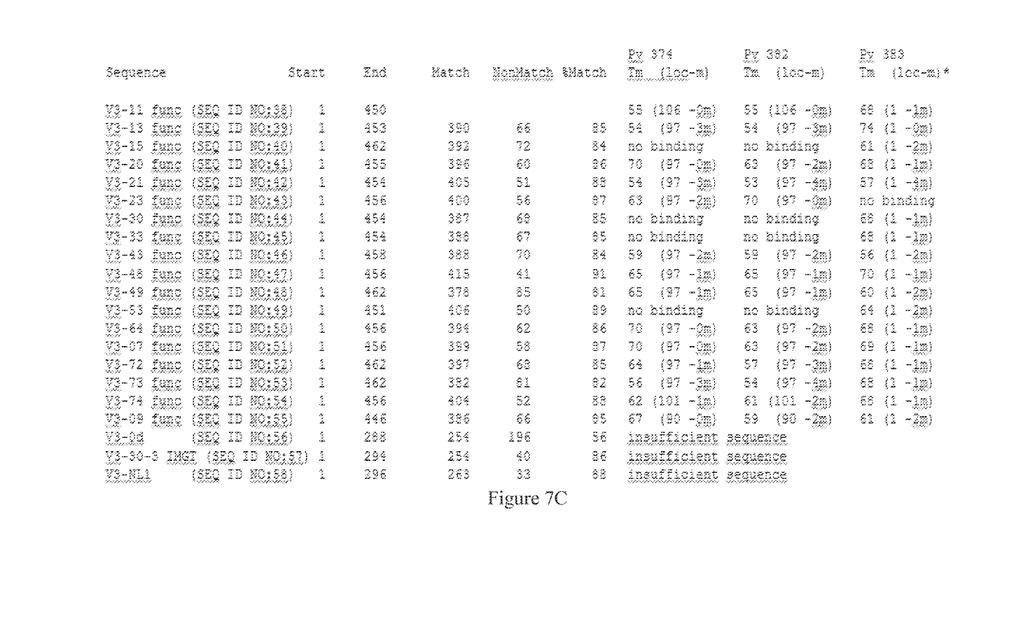

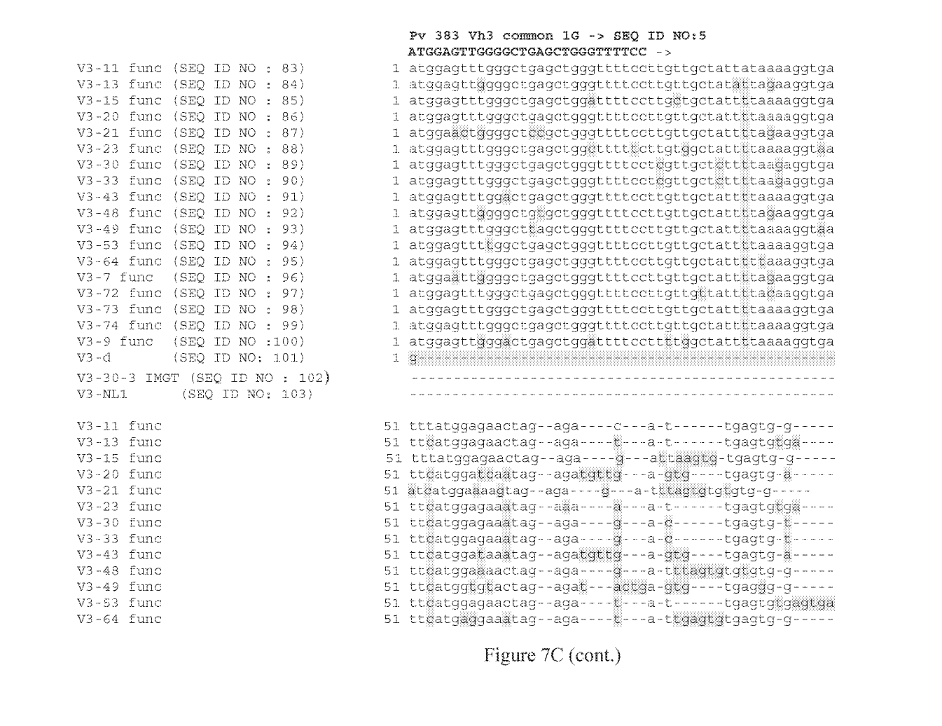

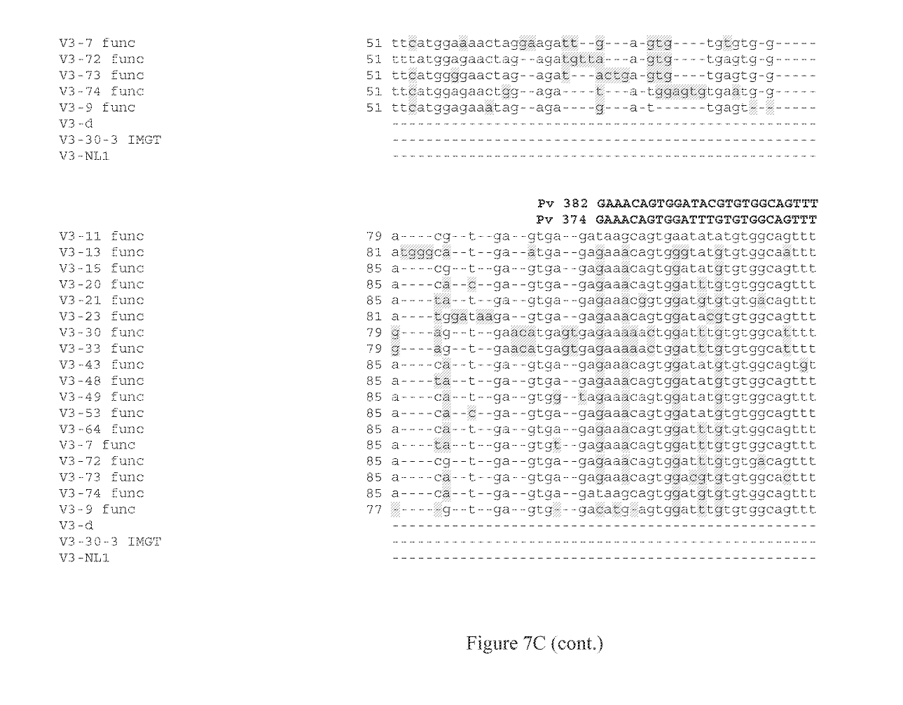

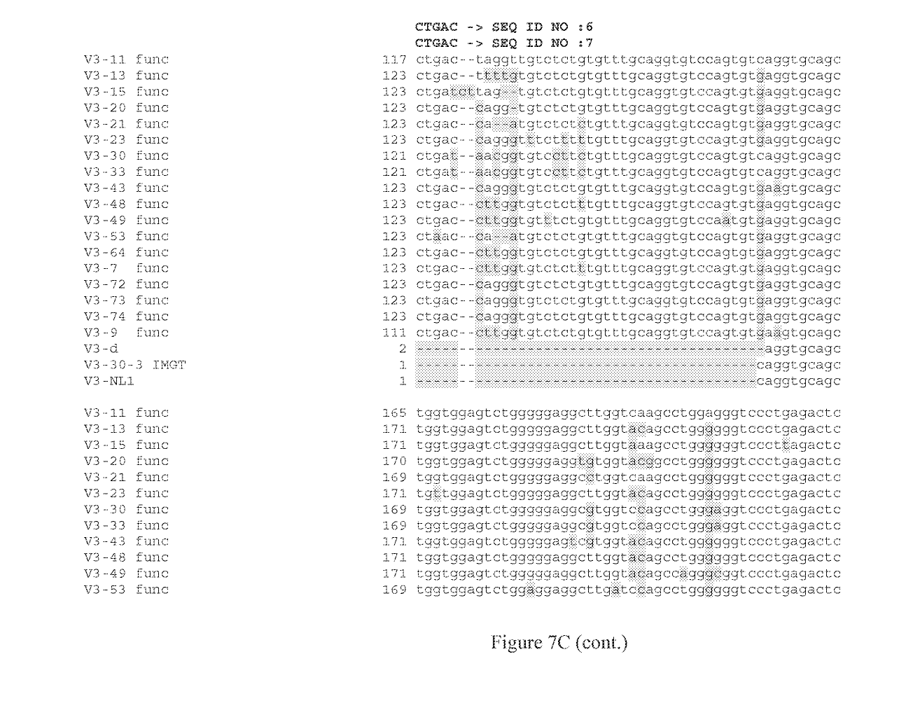

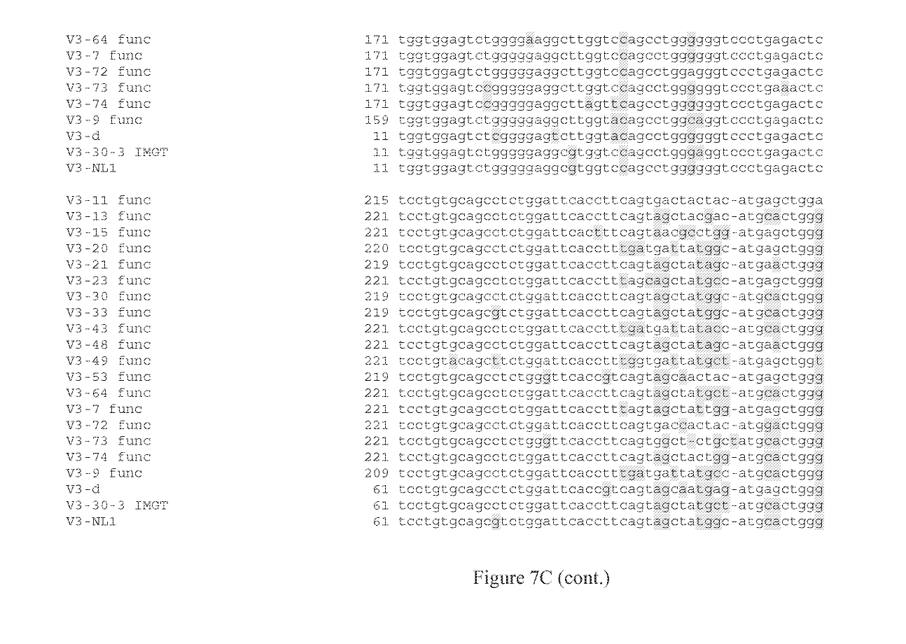

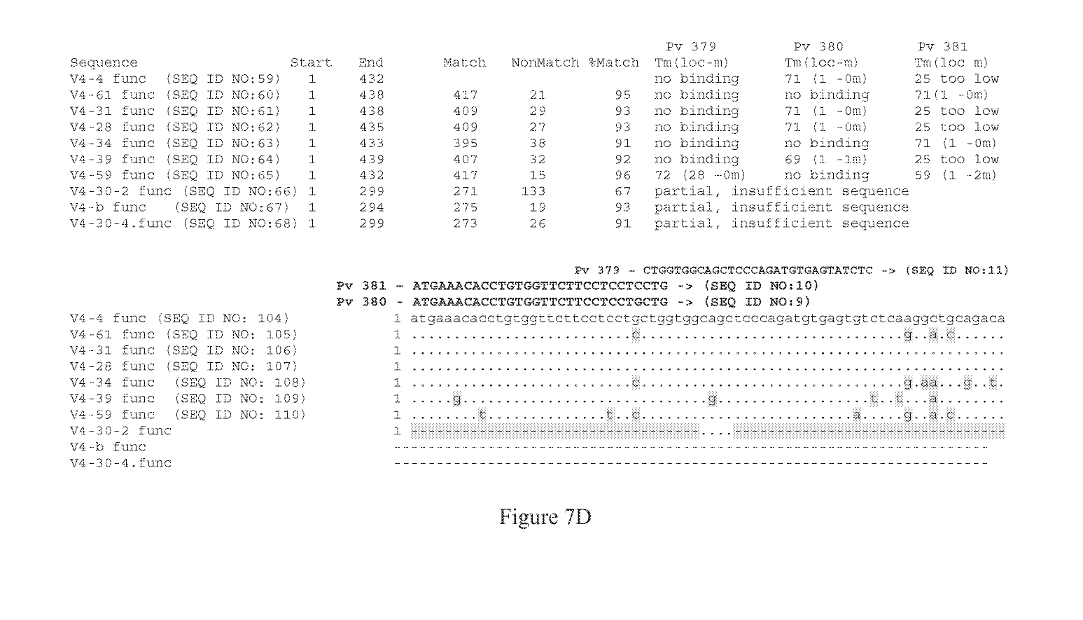

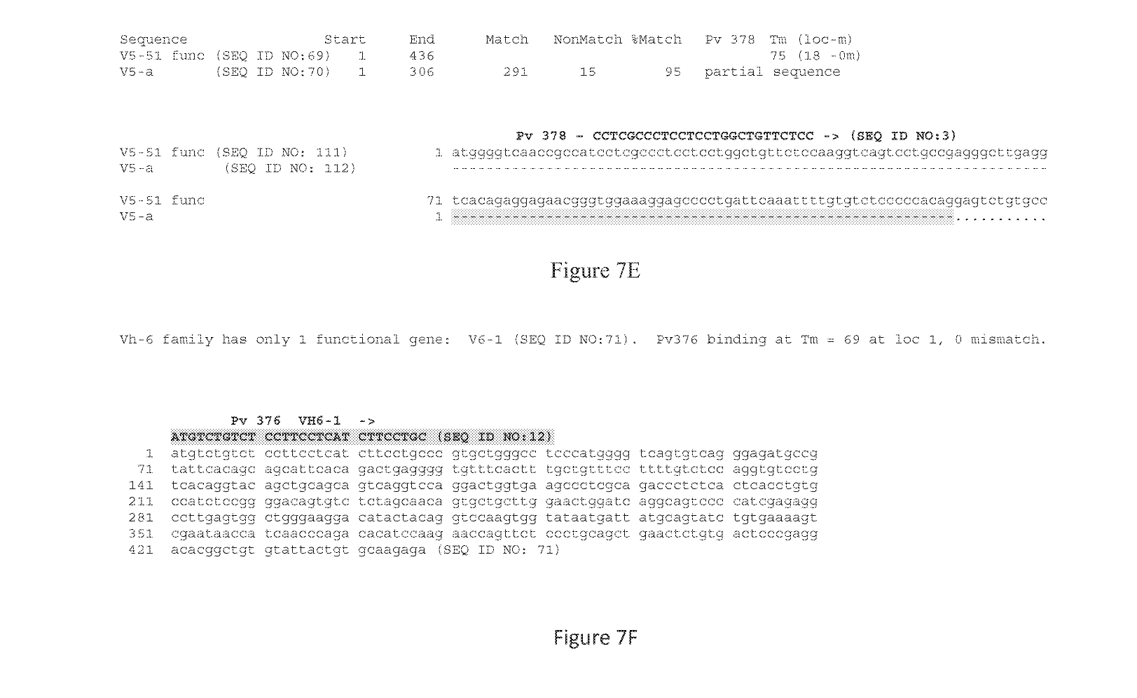

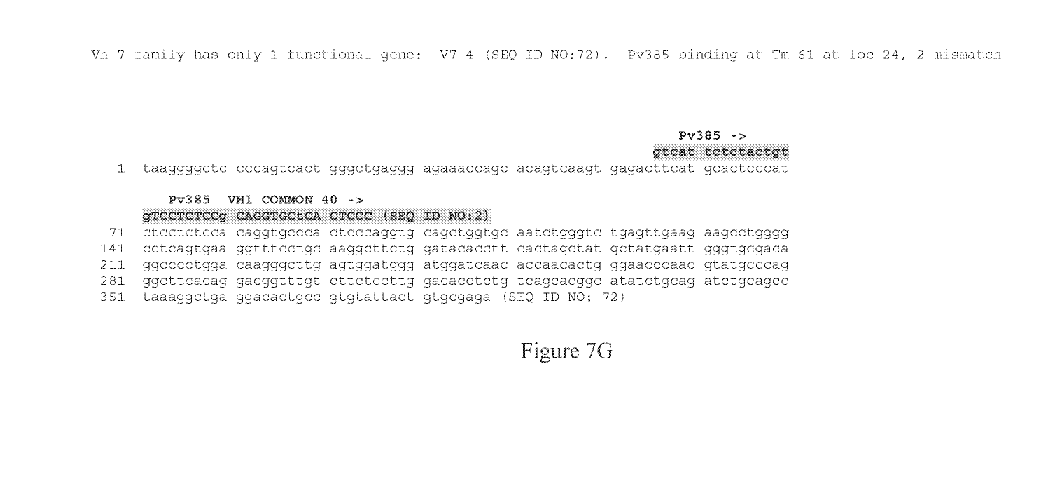

Strategy of Method development. A PCR based assay was developed that allowed analysis of the clonal IGH from CLL tumors using peripheral blood sample that was straightforward, reliable and provided the most robust mutation data possible. Preliminary studies showed that intronic Jh regions could be heavily mutated in IGH from B-cell tumors, suggesting these regions might provide an unbiased record of activation-induced cytidine deaminase (AID) activity, as compared to Vh regions which must encode a functional protein, presumably capable of interacting with antigen. The overall design to amplify this extended region of the IGH region, to include the Vh, V(D)J junction and downstream intronic regions, was achieved by designing Vh-family consensus primers (5′ end) to pair with a downstream primer located ˜1 kb downstream of Jh-6, the most 3′ of the functional J genes (FIG. 1). To limit the effects of mutation on primer binding sites, a priority was made to identify 3′ Vh-primers as close to the promoter as possible while placing the single 5′ primer beyond the AID activity window, which starts approximately 300 bases upstream and ends approximately 850 bases downstream of the V(D)J junction. The primer binding sites and Tm of the primers for Vh families are shown in FIG. 7. To limit amplification of off-target products, all primers were designed to function with stringent PCR conditions, with most having Tm>65° C. and limited mispriming to alternate sites on the human genome as determined using the Synahybridise microarray design probe verification analysis tools (version 1.0.4) from the Malaysian Genomics Resource Centre.

Results

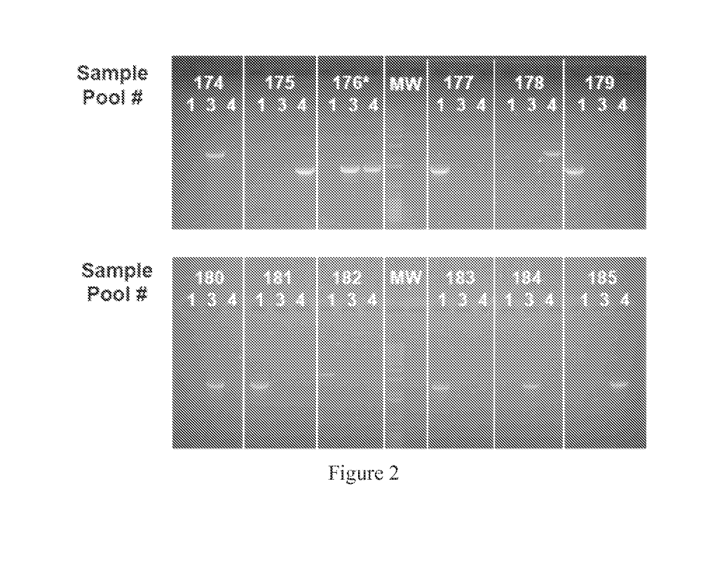

The primers were divided into 3 master mixes (i.e., primer pools) to separate the high-usage gene family primers (Vh1, Vh3 and Vh4) and minimize the probability that CLL specimens with 2 rearranged IGH alleles would generate both amplicons in the same master mix (Pool 1, 3, or 4). Of the 55 samples, 27 had a Vh region % ID≧98% to reference, while 28 samples were <98% ID, implying that SHM had occurred in these samples. In the 55 samples tested, 7 samples had both alleles rearranged resulting in 2 products from these samples. In 5 of those cases, the appropriate amplicons were in separate PCR reactions (FIG. 2), while the other 2 required additional PCR (deconstruction of the primer pool) or gel purification to resolve the 2 amplicons for sequencing. In all 7 cases with 2 rearranged alleles, one was found to be functional while the other was not capable of being expressed.

A robust PCR based assay from DNA isolated from peripheral blood of CLL patients for the simultaneous determination of both Vh and intronic-J sequence analyses was developed. IGH was isolated from Vh families 1,3,4,5,and 6, including the clinically significant 3-21 and 4-34, and the over-represented 1-69. The LE primer set directly yielded sequence-ready amplicons in 48/55 samples. Seven specimens had both alleles rearranged, in which 5 sets were separated by deconstruction of the multiplex primer set, and 2 required gel purification. No cases required cloning to obtain clean sequence results.

Further, the %ID to reference was very highly correlated in Vh and J-intronic sequences but only specimens with mutated Vh<98% ID had Intronic J-region indels≧6 bases. Vh % ID and Intronic-J region % ID results were discordant in 5/55 cases: 2 samples with 99%>Vh≧98% ID and Intronic-J<98% ID, while 3 samples with Vh=98% ID have Intronic-J>98% ID, indicating that evaluating both regions can clarify CLL mutation status.

1. A method of predicting the outcome of a chronic lymphocytic leukemia (CLL) in a subject, the method comprising:
(a) performing a polymerase chain reaction (PCR) assay for an immunoglobulin heavy (IGH) locus on a nucleic acid from a biological sample from a subject with CLL, wherein the PCR assay amplifies a non-coding region of the IGH locus;
(b) sequencing a product from the PCR assay; and
(c) determining a level of mutation in the non-coding region of the IGH locus, wherein an increased level of mutation in the non-coding region as compared to a control indicates a positive outcome for the subject with CLL and a decreased level of mutation or no mutation as compared to the control indicates a poor outcome for the subject with CLL.

2. The method of claim 1, wherein the sample comprises at least one selected from the group consisting of a lymph node, a paraffin embedded sample, a blood sample, a saliva sample, and a biopsy.

3. (canceled)

4. (canceled)

5. (canceled)

6. (canceled)

7. The method of claim 1, wherein the IGH locus comprises a Vh region, a diversity region, a joining region, and downstream non-coding region.

8. The method of claim 1, wherein the PCR assay is performed with at least one first primer and a second primer.

9. The method of claim 8, wherein the at least one first primer hybridizes with a Vh region.

10. (canceled)

11. (canceled)

12. The method of claim 9, wherein the Vh region is selected from the group consisting of Vh region 1, Vh region 2, Vh region 3, Vh region 4, Vh region 5, Vh region 6, and Vh region 7.

13. (canceled)

14. (canceled)

15. (canceled)

16. (canceled)

17. (canceled)

18. (canceled)

19. (canceled)

20. The method of claim 8, wherein the second primer hybridizes with a non-coding region downstream of a joining region.

21. (canceled)

22. (canceled)

23. (canceled)

24. (canceled)

25. The method of claim 8, wherein at least two first primers and the second primer are in a primer pool.

26. (canceled)

27. (canceled)

28. (canceled)

29. (canceled)

30. The method of claim 25, wherein the primer pool comprises a Vh region 1 hybridizing primer, a Vh region 5 hybridizing primer, and a Vh region 7 hybridizing primer.

31. (canceled)

32. The method of claim 25, wherein the primer pool comprises a Vh region 3 hybridizing primer and a Vh region 2 hybridizing primer.

33. (canceled)

34. The method of claim 25, wherein the primer pool comprises a Vh region 4 hybridizing primer and a Vh region 6 hybridizing primer.

35. (canceled)

36. (canceled)

37. (canceled)

38. (canceled)

39. (canceled)

40. (canceled)

41. (canceled)

42. (canceled)

43. A kit for predicting the outcome of a chronic lymphocytic leukemia (CLL) in a subject, the kit comprising:
(a) at least one first primer; and
(b) a second primer, wherein the at least one first primer and second primer are for performing a polymerase chain reaction (PCR) assay for an immunoglobulin heavy (IGH) locus on the nucleic acid, wherein the PCR assay amplifies a non-coding region of the IGH locus.

44. The kit of claim 43, wherein the IGH locus comprises a Vh region, a diversity region, a joining region, and a downstream non-coding region.

45. The kit of claim 44, wherein the at least one first primer hybridizes with a Vh region.

46. (canceled)

47. (canceled)

48. The kit of claim 45, wherein the Vh region is selected from the group consisting of Vh region 1, Vh region 2, Vh region 3, Vh region 4, Vh region 5, Vh region 6, and Vh region 7.

49. (canceled)

50. (canceled)

51. (canceled)

52. (canceled)

53. (canceled)

54. (canceled)

55. (canceled)

56. The kit of claim 44, wherein the second primer hybridizes with a non-coding region downstream of a joining region.

57. (canceled)

58. (canceled)

59. (canceled)

60. (canceled)

61. The kit of claim 44, wherein at least two first primers and the second primer are in a primer pool.

62. (canceled)

63. (canceled)

64. (canceled)

65. (canceled)

66. The kit of claim 61, wherein the primer pool comprises a Vh region 1 hybridizing primer, a Vh region 5 hybridizing primer, and a Vh region 7 hybridizing primer.

67. (canceled)

68. The kit of claim 61, wherein the primer pool comprises a Vh region 3 hybridizing primer and a Vh region 2 hybridizing primer.

69. (canceled)

70. The kit of claim 61, wherein the primer pool comprises a Vh region 4 hybridizing primer and a Vh region 6 hybridizing primer.

71. (canceled)

72. (canceled)

73. (canceled)

74. (canceled)

75. (canceled)

76. (canceled)

77. A method of treating a subject with a chronic lymphocytic leukemia (CLL), the method comprising:
(a) performing a polymerase chain reaction (PCR) assay for an immunoglobulin heavy (IGH) locus on a nucleic acid from a biological sample from a subject with CLL, wherein the PCR assay amplifies a non-coding region of the IGH locus;
(b) sequencing a product from the PCR assay;
(c) determining a low level of mutation or no mutation in the non-coding region as compared to a control; and
(d) providing to the subject at least one therapy selected from the group consisting of administration of a chemotherapeutic agent and a stem cell transplant.

78. The method of claim 77, the IGH locus comprises a Vh region, a diversity region, a joining region, and a downstream non-coding region.

79. The method of claim 77, wherein the 1⁻¹C1(assay is performed with at least one first primer and a second primer.

80. The method of claim 79, wherein the at least one first primer hybridizes with a Vh region.

81. (canceled)

82. (canceled)

83. The method of claim 80, wherein the Vh region is selected from the group consisting of Vh region 1, Vh region 2, Vh region 3, Vh region 4, Vh region 5, Vh region 6, and Vh region 7.

84. (canceled)

85. (canceled)

86. (canceled)

87. (canceled)

88. (canceled)

89. (canceled)

90. (canceled)

91. The method of claim 79, wherein the second primer hybridizes with a non-coding region downstream of a joining region.

92. (canceled)

93. (canceled)

94. (canceled)

95. (canceled)

96. The method of claim 79, wherein at least two first primers and the second primer are in a primer pool.

97. (canceled)

98. (canceled)

99. (canceled)

100. (canceled)

101. The method of claim 96, wherein the primer pool comprises a Vh region 1 hybridizing primer, a Vh region 5 hybridizing primer, and a Vh region 7 hybridizing primer.

102. (canceled)

103. The method of claim 96, wherein the primer pool comprises a Vh region 3 hybridizing primer and a Vh region 2 hybridizing primer.

104. (canceled)

105. The method of claim 96, wherein the primer pool comprises a Vh region 4 hybridizing primer and a Vh region 6 hybridizing primer.

106. (canceled)

107. (canceled)

108. (canceled)

109. (canceled)

110. (canceled)

111. (canceled)

112. (canceled)

113. (canceled)

114. (canceled)

115. (canceled)


SEQUENCE LISTING (112 sequences)

SEQ ID NO: 1 - DNA, 34 bp, Artificial sequence
  Synthetic construct
atggactggacctggagcatcctcttcttggtgg

SEQ ID NO: 2 - DNA, 40 bp, Artificial sequence
  Synthetic construct
gtcattctctactgtgtcctctccgcaggtgctcactccc

SEQ ID NO: 3 - DNA, 26 bp, Artificial sequence
  Synthetic construct
cctcgccctcctcctggctgttctcc

SEQ ID NO: 4 - DNA, 29 bp, Artificial sequence
  Synthetic construct
cttcttgatggcagcagcaacaggtaagg

SEQ ID NO: 5 - DNA, 28 bp, Artificial sequence
  Synthetic construct
atggagttggggctgagctgggttttcc

SEQ ID NO: 6 - DNA, 31 bp, Artificial sequence
  Synthetic construct
gaaacagtggatacgtgtggcagtttctgac

SEQ ID NO: 7 - DNA, 31 bp, Artificial sequence
  Synthetic construct
gaaacagtggatttgtgtggcagtttctgac

SEQ ID NO: 8 - DNA, 31 bp, Artificial sequence
  Synthetic construct
ttgtctcctttgtgggcttcatcttcttatg

SEQ ID NO: 9 - DNA, 30 bp, Artificial sequence
  Synthetic construct
atgaaacacctgtggttcttcctcctgctg

SEQ ID NO: 10 - DNA, 30 bp, Artificial sequence
  Synthetic construct
atgaaacacctgtggttcttcctcctcctg

SEQ ID NO: 11 - DNA, 30 bp, Artificial sequence
  Synthetic construct
ctggtggcagctcccagatgtgagtatctc

SEQ ID NO: 12 - DNA, 28 bp, Artificial sequence
  Synthetic construct
atgtctgtctccttcctcatcttcctgc

SEQ ID NO: 13 - DNA, 25 bp, Artificial sequence
  Synthetic construct
gccacctgctgtgggtgccggagac

SEQ ID NO: 14 - DNA, 3223 bp, Homo sapiens
tgaatacttccagcactggggccagggcaccctggtcaccgtctcctcaggtgagtctgctgtctggggatagcggggagccaggtgtactgggccaggcaagggctttggcttcagacttggggacaggtgctcagcaaaggaggtcggcaggagggcggagggtgtgtttttgtatgggagaagcaggagggcagaggctgtgctactggtacttcgatctctggggccgtggcaccctggtcactgtctcctcaggtgagtcccactgcagccccctcccagtcttctctgtccaggcaccaggccaggtatctggggtctgcagccggcctgggtctggcctgaggccacaccagctgccatccctggggtctccgccatgggctgcatgccagagccctgctgtcacttagccctggggccagctggagcccccaaggacaggcagggaccccgctgggcttcagccccgtcagggaccctccacaggtagcaagcaggccgagggcagggacgggaaggagaagttgtgggcagagcctgggctggggctgggcgctggctgttcatgtgccggggaccaggcctgcgctttagtgtggctacaagtgcttggagcactggggccagggcagcccggccaccgtctccctgggaacgtcacccctccctgcctgggtctcagcccgggggtctgtgtggctggggacagggacgccggctgcctctgctctgtgcttgggccatgtgacccattcgagtgtcctgcacgggcacaggtttgtgtctgggcaggaacagggactgtgtccctgtgtgatgcttttgatatctggggccaagggacaatggtcaccgtctcttcaggtaagatggctttccttctgcctcctttctctgggcccagcgtcctctgtcctggagctgggagataatgtccgggggctccttggtctgcgctgggccatgtggggccctccggggctccttctccggctgtttgggaccacgttcagcagaaggcctttctttgggaactgggactctgctgctggggcaaagggtgggcagagtcatgcttgtgctggggacaaaatgaccttgggacacggggctggctgccacggccggcccgggacagtcggagagtcaggtttttgtgcaccccttaatggggcctcccacaatgtgactactttgactactggggccagggaaccctggtcaccgtctcctcaggtgagtcctcacaacctctctcctgctttaactctgaagggttttgctgcatttctggggggaaataagggtgctgggtctcctgccaagagagccccggagcatcctggggggctcaggaggatgccctgaggcaacagcggccacacagacgaggggcaagggctccagatgctccttcctcctgagcccagcagcacgggtctctctgtggccagggccaccctaggcctctggggtccaatgcccaacaacccccgggccctccccgggctcagtctgagagggtcccagggacgtagcggggcgccagttcttgcctggggtcctggcattgttgtcacaatgtgacaactggttcgacccctggggccagggaaccctggtcaccgtctcctcaggtgagtcctcaccaccccctctctgagtccacttagggagactcagcttgccagggtctcagggtcagagtcttggaggcattttggaggtcaggaaagaaagccggggagagggacccttcgaatgggaacccagcctgtcctccccaagtccggccacagatgtcggcagctggggggctccttcggctggtctggggtgacctctctccgcttcacctggagcattctcaggggctgtcgtgatgattgcgtggtgggactctgtcccgctccaaggcacccgctctctgggacgggtgccccccggggtttttggactcctgggggtgacttagcagccgtctgcttgcagttggacttcccaggccgacagtggtctggcttctgaggggtcaggccagaatgtggggtacgtgggaggccagcagagggttccatgagaagggcaggacagggccacggacagtcagcttccatgtgacgcccggagacagaaggtctctgggtggctgggtttttgtggggtgaggatggacattctgccattgtgattactactactactactacatggacgtctggggcaaagggaccacggtcaccgtctcctcaggtaagaatggccactctagggcctttgttttctgctactgcctgtggggtttcctgagcattgcaggttggtcctcggggcatgttccgaggggacctgggcggactggccaggaggggacgggcactggggtgccttgaggatctgggagcctctgtggattttccgatgcctttggaaaatgggactcaggttgggtgcgtctgatggagtaactgagcctgggggcttggggagccacatttggacgagatgcctgaacaaaccaggggtcttagtgatggctgaggaatgtgtctcaggagcggtgtctgtaggactgcaagatcgctgcacagcagcgaatcgtgaaatattttctttagaattatgaggtgcgctgtgtgtcaacctgcatcttaaattctttattggctggaaagagaactgtcggagtgggtgaatccagccaggagggacgcgtagccccggtcttgatgagagcagggttgggggcaggggtagcccagaaacggtggctgccgtcctgacaggggcttagggaggctccaggacctcagtgccttgaagctggtttccaagagaaaaggattgtttatcttaggaggcatgcttactgttaaaagacaggatatgtttgaagtggcttctgagaaaaatggttaagaaaattatgacttaaaaatgtgagagattttcaagtatattaatttttttaactgtccaagtatttgaaattcttatcatttgattaacacccatgagtgatatgtgtctggaattgaggccaaagcaagctcagctaagaaatactagcacagtgctgtcggccccgatgcgggactgcgttttgaccatcataaatcaagtttatttttttaattaattgagcgaagctggaagcagatgatgaattagagtcaagatggctgcatgggggtctccggcacccacagcaggtggc

SEQ ID NO: 15 - DNA, 3225 bp, homo sapiens
gctgaatacttccagcactggggccagggcaccctggtcaccgtctcctcaggtgagtctgctgtctggggatagcggggagccaggtgtactgggccaggcaagggctttggcttcagacttggggacaggtgctcagcaaaggaggtcggcaggagggcggagggtgtgtttttgtatgggagaagcaggagggcagaggctgtgctactggtacttcgatctctggggccgtggcaccctggtcactgtctcctcaggtgagtcccactgcagccccctcccagtcttctctgtccaggcaccaggccaggtatctggggtctgcagccggcctgggtctggcctgaggccacaccagctgccatccctggggtctccgccatgggctgcatgccagagccctgctgtcacttagccctggggccagctggagcccccaaggacaggcagggaccccgctgggcttcagccccgtcagggaccctccacaggtagcaagcaggccgagggcagggacgggaaggagaagttgtgggcagagcctgggctggggctgggcgctggctgttcatgtgccggggaccaggcctgcgctttagtgtggctacaagtgcttggagcactggggccagggcagcccggccaccgtctccctgggaacgtcacccctccctgcctgggtctcagcccgggggtctgtgtggctggggacagggacgccggctgcctctgctctgtgcttgggccatgtgacccattcgagtgtcctgcacgggcacaggtttgtgtctgggcaggaacagggactgtgtccctgtgtgatgcttttgatrtctggggccaagggacaatggtcaccgtctcttcaggtaagatggctttccttctgcctcctttctctgggcccagcgtcctctgtcctggagctgggagataatgtccgggggctccttggtctgcgctgggccatgtggggccctccggggctccttctccggctgtttgggaccacgttcagcagaaggcctttctttgggaactgggactctgctgctggggcaaagggtgggcagagtcatgcttgtgctggggacaaaatgaccttgggacacggggctggctgccacggccggcccgggacagtcggagagtcaggtttttgtgcaccccttaatggggcctcccacaatgtgrctactttgactactggggccarggraccctggtcaccgtctcctcaggtgagtcctcacaacctctctcctgctttaactctgaagggttttgctgcatttytggggggaaataagggtgctgggtctcctgccaagagagccccggagcakcctggggggctcaggaggatgccctgaggcaacagcggccacacagacgaggggcaagggctccagatgctccttcctcctgagcccagcagcacgggtctctctgtggccagggccaccctaggcctctggggtccaatgcccaacaacccccgggccctccccgggctcagtctgagagggtcccagggacgtagcggggcgccagttcttgcctggggtcctggcattgttgtcacaatgtgacaactggttcgacycctggggccarggaaccctggtcaccgtctcctcaggtgagtcctcaccaccccctctctgagtccacttagggagactcagcttgccagggtctcagggtcagagtcttggaggcattttggaggtcaggaaagaaagcyggggagagggacccttcgaatgggaacccagcctgtcctccccaagtccggccacagatgtcggcagctggggggctccttcggctggtctggggtgacctctctccgcttcacctggagcattctcaggggctgtcgtgatgattgcgtggtgggactctgtcccgctccaaggcacccgctctctgggacgggtgccccccggggtttttggactcctgggggtgacttagcagccgtctgcttgcagttggacttcccaggccgacagtggtctggcttctgaggggtcaggccagaatgtggggtacgtgggaggccagcagagggttccatgagaagggcaggacagggccacggacagtcagcttccatgtgacgcccggagacagaaggtctctgggtggctgggtttttgtggggtgaggatggacattctgccattgtgattactactactactackryatggacgtctggggsmaagggaccacggtcaccgtctcctcaggtaagaatggccactctagggcctttgttttctgctactgcctgtggggtttcctgagcattgcaggttggtcctcggggcatgttccgaggggacctgggcggactggccaggaggggakgggcactggggtgccttgaggatctgggagcctctgtggattttccgatgcctttggaaaatgggactcaggttgggtgcgtctgatggagtaactgagcctgggggcttggggagccacatttggacgagatgcctgaacaaaccaggggtcttagtgatggctgaggaatgtgtctcaggagcggtgtctgtaggactgcaagatcgctgcacagcagcgaatcgtgaaatattttctttagaattatgaggtgcgctgtgtgtcaacctgcatcttaaattctttattggctggaaagagaactgtcggagtgggtgaatccagccaggagggacgcgtagccccggtcttgatgagagcagggttgggggcaggggtagcccagaaacggtggctgccgtcctgacaggggcttagggaggctccaggacctcagtgccttgaagctggtttccaagagaaaaggattgtttatcttaggaggcatgcttactgttaaaagacaggatatgtttgaagtggcttctgagaaaaatggttaagaaaattatgacttaaaaatgtgagagattttcaagtatattaatttttttaactgtccaagtatttgaaattcttatcatttgattaacacccatgagtgatatgtgtctggaattgaggccaaagcaagctcagctaagaaatactagcacagtgctgtcggccccgatgcgggactgcgttttgaccatcataaatcaagtttatttttttaattaattgagcgaagctggaagcagatgatgaattagagtcaagatggctgcatgggggtctccggcacccacagcaggtggc

SEQ ID NO: 16 - DNA, 18 bp, Artificial sequence
  Synthetic construct
gctgtgggtgccggagac

SEQ ID NO: 17 - DNA, 25 bp, Artificial sequence
  Synthetic construct
cgcccaggtcccctcggaacatgcc

SEQ ID NO: 18 - DNA, 18 bp, Artificial sequence
  Synthetic construct
aggtcccctcggaacatg

SEQ ID NO: 19 - DNA, 20 bp, Artificial sequence
  Synthetic construct
gcctttgttttctgctactg

SEQ ID NO: 20 - DNA, 18 bp, Artificial sequence
  Synthetic construct
ctgggttcccattcgaag

SEQ ID NO: 21 - DNA, 18 bp, Artificial sequence
  Synthetic construct
tgctccggggctctcttg

SEQ ID NO: 22 - DNA, 18 bp, Artificial sequence
  Synthetic construct
ccaaacagccggagaagg

SEQ ID NO: 23 - DNA, 18 bp, Artificial sequence
  Synthetic construct
gccccagggctaagtgac

SEQ ID NO: 24 - DNA, 18 bp, Artificial sequence
  Synthetic construct
ctgaagccaaagcccttg

SEQ ID NO: 25 - DNA, 438 bp, Homo sapiens
atggactggatttggagggtcctcttcttggtgggagcagcgacaggcaaggagatgccaagtcccagtgatgaggaggggattgagtccagtcaaggtggctttcatccactcctgtgttctctccacaggtgcccactcccaaatgcagctggtgcagtctgggcctgaggtgaagaagcctgggacctcagtgaaggtctcctgcaaggcttctggattcacctttactagctctgctatgcagtgggtgcgacaggctcgtggacaacgccttgagtggataggatggatcgtcgttggcagtggtaacacaaactacgcacagaagttccaggaaagagtcaccattaccagggacatgtccacaagcacagcctacatggagctgagcagcctgagatccgaggacacggccgtgtattactgtgcggcaga

SEQ ID NO: 26 - DNA, 438 bp, Homo sapiens
atggactggacctggagaatcctcttcttggtggcagcagccacaggtaaggggctcccaagtcccagtgatgaggaggggattgagtccagtcaaggtggcttttatccactcctgtgtcccctccacagatgcctactcccagatgcagctggtgcagtctggggctgaggtgaagaagactgggtcctcagtgaaggtttcctgcaaggcttccggatacaccttcacctaccgctacctgcactgggtgcgacaggcccccggacaagcgcttgagtggatgggatggatcacacctttcaatggtaacaccaactacgcacagaaattccaggacagagtcaccattaccagggacaggtctatgagcacagcctacatggagctgagcagcctgagatctgaggacacagccatgtattactgtgcaagata

SEQ ID NO: 27 - DNA, 438 bp, Homo sapiens
atggactggacctggaggatcctcttcttggtggcagcagccacaggtaagaggctccctagtcccagtgatgagaaagagattgagtccagtccagggagatctcatccacttctgtgttctctccacaggagcccactcccaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagtttcagggcagggtcaccatgaccagggacacgtccatcagcacagcctacatggagctgagcaggctgagatctgacgacacggccgtgtattactgtgcgagaga

SEQ ID NO: 28 - DNA, 437 bp, Homo sapiens
atggactggacctggagcatccttttcttggtggcagcagcaacaggtaacggactccccagtcccagggctgagagagaaaccaggccagtcatgtgagacttcacccactcctgtgtcctctccacaggtgcccactcccaggttcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggccgtgtattactgtgcgagaga

SEQ ID NO: 29 - DNA, 439 bp, Homo sapiens
atggactggacctggaggttcctctttgtggtggcagcagctacaggtaaggggcttcctagtcctaaggctgaggaagggatcctggtttagttaaagaggattttattcacccctgtgtcctctccacaggtgtccagtcccaggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacaaatccacgagcacagcctacatggagctgagcagcctgagatctgaggacacggccgtgtattactgtgcgagaga

SEQ ID NO: 30 - DNA, 439 bp, Homo sapiens
atggactggacctggaggatcctcttcttggtggcagcagctacaagtaaggggcttcctagtctcaaagctgaggaacggatcctggttcagtcaaagaggattttattctctcctgtgttctctccacaggtgcccactcccaggtgcagctggtgcagtctggggctgaggtgaagaagcctggggcctcagtgaaggtctcctgcaaggcttctggatacaccttcaccagttatgatatcaactgggtgcgacaggccactggacaagggcttgagtggatgggatggatgaaccctaacagtggtaacacaggctatgcacagaagttccagggcagagtcaccatgaccaggaacacctccataagcacagcctacatggagctgagcagcctgagatctgaggacacggccgtgtattactgtgcgagagg

SEQ ID NO: 31 - DNA, 294 bp, Homo sapiens
gaggtccagctggtacagtctggggctgaggtgaagaagcctggggctacagtgaaaatctcctgcaaggtttctggatacaccttcaccgactactacatgcactgggtgcaacaggcccctggaaaagggcttgagtggatgggacttgttgatcctgaagatggtgaaacaatatacgcagagaagttccagggcagagtcaccataaccgcggacacgtctacagacacagcctacatggagctgagcagcctgagatctgaggacacggccgtgtattactgtgcaaca

SEQ ID NO: 32 - DNA, 438 bp, Homo sapiens
atggactgcacctggaggatcctcttcttggtggcagcagctacaggcaagagaatcctgagttccagggctgatgaggggactgggtccagttaagtggtgtctcatccactcctctgtcctctccacaggcacccacgcccaggtccagctggtacagtctggggctgaggtgaagaagcctggggcctcagtgaaggtctcctgcaaggtttccggatacaccctcactgaattatccatgcactgggtgcgacaggctcctggaaaagggcttgagtggatgggaggttttgatcctgaagatggtgaaacaatctacgcacagaagttccagggcagagtcaccatgaccgaggacacatctacagacacagcctacatggagctgagcagcctgagatctgaggacacggccgtgtattactgtgcaacaga

SEQ ID NO: 33 - DNA, 438 bp, Homo sapiens
atggactggacctggagggtcttctgcttgctggctgtagctccaggtaaagggccaactggttccagggctgaggaagggattttttccagtttagaggactgtcattctctactgtgtcctctccgcaggtgctcactcccaggtgcagctggtgcagtctggggctgaggtgaagaagcctggggcctcagtgaaggtttcctgcaaggcatctggatacaccttcaccagctactatatgcactgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgagaga

SEQ ID NO: 34 - DNA, 438 bp, Homo sapiens
atggactggacctggaggatcctctttttggtggcagcagccacaggtaaggggctgccaaatcccagtgaggaggaagggatcgaagccagtcaagggggcttccatccactcctgtgtcttctctacaggtgtccactcccaggttcagctggtgcagtctggggctgaggtgaagaagcctggggcctcagtgaaggtttcctgcaaggcttctggatacaccttcactagctatgctatgcattgggtgcgccaggcccccggacaaaggcttgagtggatgggatggagcaacgctggcaatggtaacacaaaatattcacaggagttccagggcagagtcaccattaccagggacacatccgcgagcacagcctacatggagctgagcagcctgagatctgaggacatggctgtgtattactgtgcgagaga

SEQ ID NO: 35 - DNA, 444 bp, Homo sapiens
atggacatactttgttccacgctcctgctactgactgtcccgtcctgtgagtgctgtggtcaggtagtacttcagaagcaaaaaatctattctctcctttgtgggcttcatcttcttatgtcttctccacaggggtcttatcccaggtcaccttgagggagtctggtcctgcgctggtgaaacccacacagaccctcacactgacctgcaccttctctgggttctcactcagcactagtggaatgtgtgtgagctggatccgtcagcccccagggaaggccctggagtggcttgcactcattgattgggatgatgataaatactacagcacatctctgaagaccaggctcaccatctccaaggacacctccaaaaaccaggtggtccttacaatgaccaacatggaccctgtggacacagccacgtattattgtgcacggatac

SEQ ID NO: 36 - DNA, 444 bp, Homo sapiens
atggacacactttgctccacgctcctgctgctgaccatcccttcatgtgagtgctgtggtcagggactccttcacgggtgaaacatcagttttcttgtttgtgggcttcatcttcttatgctttctccacaggggtcttgtcccagatcaccttgaaggagtctggtcctacgctggtgaaacccacacagaccctcacgctgacctgcaccttctctgggttctcactcagcactagtggagtgggtgtgggctggatccgtcagcccccaggaaaggccctggagtggcttgcactcatttattggaatgatgataagcgctacagcccatctctgaagagcaggctcaccatcaccaaggacacctccaaaaaccaggtggtccttacaatgaccaacatggaccctgtggacacagccacatattactgtgcacacagac

SEQ ID NO: 37 - DNA, 444 bp, Homo sapiens
atggacacactttgctacacactcctgctgctgaccaccccttcctgtgagtgctgtggtcagggacttcctcagaagtgaaacatcagttgtctcctttgtgggcttcatcttcttatgtcttctccacaggggtcttgtcccaggtcaccttgaaggagtctggtcctgtgctggtgaaacccacagagaccctcacgctgacctgcaccgtctctgggttctcactcagcaatgctagaatgggtgtgagctggatccgtcagcccccagggaaggccctggagtggcttgcacacattttttcgaatgacgaaaaatcctacagcacatctctgaagagcaggctcaccatctccaaggacacctccaaaagccaggtggtccttaccatgaccaacatggaccctgtggacacagccacatattactgtgcacggatac

SEQ ID NO: 38 - DNA, 450 bp, Homo sapiens
atggagtttgggctgagctgggttttccttgttgctattataaaaggtgatttatggagaactagagacattgagtggacgtgagtgagataagcagtgaatatatgtggcagtttctgactaggttgtctctgtgtttgcaggtgtccagtgtcaggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattagtagtagtggtagtaccatatactacgcagactctgtgaagggccgattcaccatctccagggacaacgccaagaactcactgtatctgcaaatgaacagcctgagagccgaggacacggccgtgtattactgtgcgagaga

SEQ ID NO: 39 - DNA, 453 bp, Homo sapiens
atggagttggggctgagctgggttttccttgttgctatattagaaggtgattcatggagaactagagatattgagtgtgaatgggcatgaatgagagaaacagtgggtatgtgtggcaatttctgacttttgtgtctctgtgtttgcaggtgtccagtgtgaggtgcagctggtggagtctgggggaggcttggtacagcctggggggtccctgagactctcctgtgcagcctctggattcaccttcagtagctacgacatgcactgggtccgccaagctacaggaaaaggtctggagtgggtctcagctattggtactgctggtgacacatactatccaggctccgtgaagggccgattcaccatctccagagaaaatgccaagaactccttgtatcttcaaatgaacagcctgagagccggggacacggctgtgtattactgtgcaagaga

SEQ ID NO: 40 - DNA, 462 bp, Homo sapiens
atggagtttgggctgagctggattttccttgctgctattttaaaaggtgatttatggagaactagagagattaagtgtgagtggacgtgagtgagagaaacagtggatatgtgtggcagtttctgatcttagtgtctctgtgtttgcaggtgtccagtgtgaggtgcagctggtggagtctgggggaggcttggtaaagcctggggggtcccttagactctcctgtgcagcctctggattcactttcagtaacgcctggatgagctgggtccgccaggctccagggaaggggctggagtgggttggccgtattaaaagcaaaactgatggtgggacaacagactacgctgcacccgtgaaaggcagattcaccatctcaagagatgattcaaaaaacacgctgtatctgcaaatgaacagcctgaaaaccgaggacacagccgtgtattactgtaccacaga

SEQ ID NO: 41 - DNA, 455 bp, Homo sapiens
atggagtttgggctgagctgggttttccttgttgctattttaaaaggtgattcatggatcaatagagatgttgagtgtgagtgaacacgagtgagagaaacagtggatttgtgtggcagtttctgaccaggtgtctctgtgtttgcaggtgtccagtgtgaggtgcagctggtggagtctgggggaggtgtggtacggcctggggggtccctgagactctcctgtgcagcctctggattcacctttgatgattatggcatgagctgggtccgccaagctccagggaaggggctggagtgggtctctggtattaattggaatggtggtagcacaggttatgcagactctgtgaagggccgattcaccatctccagagacaacgccaagaactccctgtatctgcaaatgaacagtctgagagccgaggacacggccttgtatcactgtgcgagaga

SEQ ID NO: 42 - DNA, 454 bp, Homo sapiens
atggaactggggctccgctgggttttccttgttgctattttagaaggtgaatcatggaaaagtagagagatttagtgtgtgtggatatgagtgagagaaacggtggatgtgtgtgacagtttctgaccaatgtctctctgtttgcaggtgtccagtgtgaggtgcagctggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagaga

SEQ ID NO: 43 - DNA, 456 bp, Homo sapiens
atggagtttgggctgagctggctttttcttgtggctattttaaaaggtaattcatggagaaatagaaaaattgagtgtgaatggataagagtgagagaaacagtggatacgtgtggcagtttctgaccagggtttctttttgtttgcaggtgtccagtgtgaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaaga

SEQ ID NO: 44 - DNA, 454 bp, Homo sapiens
atggagtttgggctgagctgggttttcctcgttgctcttttaagaggtgattcatggagaaatagagagactgagtgtgagtgaacatgagtgagaaaaactggatttgtgtggcattttctgataacggtgtccttctgtttgcaggtgtccagtgtcaggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgagaga

SEQ ID NO: 45 - DNA, 454 bp, Homo sapiens
atggagtttgggctgagctgggttttcctcgttgctcttttaagaggtgattcatggagaaatagagagactgagtgtgagtgaacatgagtgagaaaaactggatttgtgtggcattttctgataacggtgtccttctgtttgcaggtgtccagtgtcaggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatactatgcagactccgtgaagggccgattcaccatctccagagacaattccaagaacacgctgtatctgcaaatgaacagcctgagagccgaggacacggctgtgtattactgtgcgagaga

SEQ ID NO: 46 - DNA, 458 bp, Homo sapiens
atggagtttggactgagctgggttttccttgttgctattttaaaaggtgattcatggataaatagagatgttgagtgtgagtgaacatgagtgagagaaacagtggatatgtgtggcagtgtctgaccagggtgtctctgtgtttgcaggtgtccagtgtgaagtgcagctggtggagtctgggggagtcgtggtacagcctggggggtccctgagactctcctgtgcagcctctggattcacctttgatgattataccatgcactgggtccgtcaagctccggggaagggtctggagtgggtctctcttattagttgggatggtggtagcacatactatgcagactctgtgaagggccgattcaccatctccagagacaacagcaaaaactccctgtatctgcaaatgaacagtctgagaactgaggacaccgccttgtattactgtgcaaaagata

SEQ ID NO: 47 - DNA, 456 bp, Homo sapiens
atggagttggggctgtgctgggttttccttgttgctattttagaaggtgattcatggaaaactagagagatttagtgtgtgtggatatgagtgagagaaacagtggatatgtgtggcagtttctgaccttggtgtctctttgtttgcaggtgtccagtgtgaggtgcagctggtggagtctgggggaggcttggtacagcctggggggtccctgagactctcctgtgcagcctctggattcaccttcagtagctatagcatgaactgggtccgccaggctccagggaaggggctggagtgggtttcatacattagtagtagtagtagtaccatatactacgcagactctgtgaagggccgattcaccatctccagagacaatgccaagaactcactgtatctgcaaatgaacagcctgagagacgaggacacggctgtgtattactgtgcgagaga

SEQ ID NO: 48 - DNA, 462 bp, Homo sapiens
atggagtttgggcttagctgggttttccttgttgctattttaaaaggtaattcatggtgtactagagatactgagtgtgaggggacatgagtggtagaaacagtggatatgtgtggcagtttctgaccttggtgtttctgtgtttgcaggtgtccaatgtgaggtgcagctggtggagtctgggggaggcttggtacagccagggcggtccctgagactctcctgtacagcttctggattcacctttggtgattatgctatgagctggttccgccaggctccagggaaggggctggagtgggtaggtttcattagaagcaaagcttatggtgggacaacagaatacgccgcgtctgtgaaaggcagattcaccatctcaagagatgattccaaaagcatcgcctatctgcaaatgaacagcctgaaaaccgaggacacagccgtgtattactgtactagaga

SEQ ID NO: 49 - DNA, 451 bp, Homo sapiens
atggagttttggctgagctgggttttccttgttgctattttaaaaggtgattcatggagaactagagatattgagtgtgagtgaacacgagtgagagaaacagtggatatgtgtggcagtttctaaccaatgtctctgtgtttgcaggtgtccagtgtgaggtgcagctggtggagtctggaggaggcttgatccagcctggggggtccctgagactctcctgtgcagcctctgggttcaccgtcagtagcaactacatgagctgggtccgccaggctccagggaaggggctggagtgggtctcagttatttatagcggtggtagcacatactacgcagactccgtgaagggccgattcaccatctccagagacaattccaagaacacgctgtatcttcaaatgaacagcctgagagccgaggacacggccgtgtattactgtgcgagaga

SEQ ID NO: 50 - DNA, 456 bp, Homo sapiens
atggagtttgggctgagctgggttttccttgttgctatttttaaaggtgattcatgaggaaatagagatattgagtgtgagtggacatgagtgagagaaacagtggatttgtgtggcagtttctgaccttggtgtctctgtgtttgcaggtgtccagtgtgaggtgcagctggtggagtctggggaaggcttggtccagcctggggggtccctgagactctcctgtgcagcctctggattcaccttcagtagctatgctatgcactgggtccgccaggctccagggaagggactggaatatgtttcagctattagtagtaatgggggtagcacatattatgcagactctgtgaagggcagattcaccatctccagagacaattccaagaacacgctgtatcttcaaatgggcagcctgagagctgaggacatggctgtgtattactgtgcgagaga

SEQ ID NO: 51 - DNA, 456 bp, Homo sapiens
atggaattggggctgagctgggttttccttgttgctattttagaaggtgattcatggaaaactaggaagattgagtgtgtgtggatatgagtgtgagaaacagtggatttgtgtggcagtttctgaccttggtgtctctttgtttgcaggtgtccagtgtgaggtgcagctggtggagtctgggggaggcttggtccagcctggggggtccctgagactctcctgtgcagcctctggattcacctttagtagctattggatgagctgggtccgccaggctccagggaaggggctggagtgggtggccaacataaagcaagatggaagtgagaaatactatgtggactctgtgaagggccgattcaccatctccagagacaacgccaagaactcactgtatctgcaaatgaacagcctgagagccgaggacacggctgtgtattactgtgcgagaga

SEQ ID NO: 52 - DNA, 462 bp, Homo sapiens
atggagtttgggctgagctgggttttccttgttgttattttacaaggtgatttatggagaactagagatgttaagtgtgagtggacgtgagtgagagaaacagtggatttgtgtgacagtttctgaccagggtgtctctgtgtttgcaggtgtccagtgtgaggtgcagctggtggagtctgggggaggcttggtccagcctggagggtccctgagactctcctgtgcagcctctggattcaccttcagtgaccactacatggactgggtccgccaggctccagggaaggggctggagtgggttggccgtactagaaacaaagctaacagttacaccacagaatacgccgcgtctgtgaaaggcagattcaccatctcaagagatgattcaaagaactcactgtatctgcaaatgaacagcctgaaaaccgaggacacggccgtgtattactgtgctagaga

SEQ ID NO: 53 - DNA, 462 bp, Homo sapiens
atggagtttgggctgagctgggttttccttgttgctattttaaaaggtgattcatggggaactagagatactgagtgtgagtggacatgagtgagagaaacagtggacgtgtgtggcactttctgaccagggtgtctctgtgtttgcaggtgtccagtgtgaggtgcagctggtggagtccgggggaggcttggtccagcctggggggtccctgaaactctcctgtgcagcctctgggttcaccttcagtggctctgctatgcactgggtccgccaggcttccgggaaagggctggagtgggttggccgtattagaagcaaagctaacagttacgcgacagcatatgctgcgtcggtgaaaggcaggttcaccatctccagagatgattcaaagaacacggcgtatctgcaaatgaacagcctgaaaaccgaggacacggccgtgtattactgtactagaca

SEQ ID NO: 54 - DNA, 456 bp, Homo sapiens
atggagtttgggctgagctgggttttccttgttgctattttaaaaggtgattcatggagaactggagatatggagtgtgaatggacatgagtgagataagcagtggatgtgtgtggcagtttctgaccagggtgtctctgtgtttgcaggtgtccagtgtgaggtgcagctggtggagtccgggggaggcttagttcagcctggggggtccctgagactctcctgtgcagcctctggattcaccttcagtagctactggatgcactgggtccgccaagctccagggaaggggctggtgtgggtctcacgtattaatagtgatgggagtagcacaagctacgcggactccgtgaagggccgattcaccatctccagagacaacgccaagaacacgctgtatctgcaaatgaacagtctgagagccgaggacacggctgtgtattactgtgcaagaga

SEQ ID NO: 55 - DNA, 446 bp, Homo sapiens
atggagttgggactgagctggattttccttttggctattttaaaaggtgattcatggagaaatagagagattgagtgtgagtggacatgagtggatttgtgtggcagtttctgaccttggtgtctctgtgtttgcaggtgtccagtgtgaagtgcagctggtggagtctgggggaggcttggtacagcctggcaggtccctgagactctcctgtgcagcctctggattcacctttgatgattatgccatgcactgggtccggcaagctccagggaagggcctggagtgggtctcaggtattagttggaatagtggtagcataggctatgcggactctgtgaagggccgattcaccatctccagagacaacgccaagaactccctgtatctgcaaatgaacagtctgagagctgaggacacggccttgtattactgtgcaaaagata

SEQ ID NO: 56 - DNA, 288 bp, Homo sapiens
gaggtgcagctggtggagtctcggggagtcttggtacagcctggggggtccctgagactctcctgtgcagcctctggattcaccgtcagtagcaatgagatgagctgggtccgccaggctccagggaagggtctggagtgggtctcatccattagtggtggtagcacatactacgcagactccaggaagggcagattcaccatctccagagacaattccaagaacacgctgcatcttcaaatgaacagcctgagagctgaggacacggctgtgtattactgtaagaaa

SEQ ID NO: 57 - DNA, 294 bp, Homo sapiens
caggtgcagctggtggagtctgggggaggcgtggtccagcctgggaggtccctgagactctcctgtgcagcctctggattcaccttcagtagctatgctatgcactgggtccgccaggctccaggcaaggggctggagtgggtggcagttatatcatatgatggaagcaataaatactacgcagactccgtgaagggccgattcaccatctccagagacaattccaagaacacgctgtatctgcaaatgaacagcctgagagctgaggacacggctgtgtattactgtgcgaga

SEQ ID NO: 58 - DNA, 296 bp, Homo sapiens
caggtgcagctggtggagtctgggggaggcgtggtccagcctggggggtccctgagactctcctgtgcagcgtctggattcaccttcagtagctatggcatgcactgggtccgccaggctccaggcaaggggctggagtgggtctcagttatttatagcggtggtagtagcacatactatgcagactccgtgaagggccgattcaccatctccagagacaattccaagaacacgctgtatctgcaaatgaacagcctgagagctgaggacacggctgtgtattactgtgcgaaaga

SEQ ID NO: 59 - DNA, 432 bp, Homo sapiens
atgaaacacctgtggttcttcctcctgctggtggcagctcccagatgtgagtgtctcaaggctgcagacatggggatatgggaggtgcctctgatcccagggctcactgtgggtctctctgttcacaggggtcctgtcccaggtgcagctgcaggagtcgggcccaggactggtgaagccttcggagaccctgtccctcacctgcactgtctctggtggctccatcagtagttactactggagctggatccggcagcccgccgggaagggactggagtggattgggcgtatctataccagtgggagcaccaactacaacccctccctcaagagtcgagtcaccatgtcagtagacacgtccaagaaccagttctccctgaagctgagctctgtgaccgccgcggacacggccgtgtattactgtgcgagaga

SEQ ID NO: 60 - DNA, 438 bp, Homo sapiens
atgaaacacctgtggttcttcctcctcctggtggcagctcccagatgtgagtgtctcagggatccagacatgggggtatgggaggtgcctctgatcccagggctcactgtgggtctctctgttcacaggggtcctgtcccaggtgcagctgcaggagtcgggcccaggactggtgaagccttcggagaccctgtccctcacctgcactgtctctggtggctccgtcagcagtggtggttactactggagctggatccggcagcccccagggaagggactggagtggattgggtatatctattacagtgggagcaccaactacaacccctccctcaagagtcgagtcaccatatcagtagacacgtccaagaaccagttctccctgaagctgagctctgtgaccgctgcggacacggccgtgtattactgtgcgagaga

SEQ ID NO: 61 - DNA, 438 bp, Homo sapiens
atgaaacacctgtggttcttcctcctgctggtggcagctcccagatgtgagtgtctcaaggctgcagacatggagatatgggaggtgcctctgatcccagggctcactgtgtgtctctctgttcacaggggtcctgccccaggtgcagctgcaggagtcgggcccaggactggtgaagccttcacagaccctgtccctcacctgtactgtctctggtggctccatcagcagtggtggttactactggagctggatccgccagcacccagggaagggcctggagtggattgggtacatctattacagtgggagcacctactacaacccgtccctcaagagtcgagttaccatatcagtagacacgtctaagaaccagttctccctgaagctgagctctgtgactgccgcggacacggccgtgtattactgtgcgagaga

SEQ ID NO: 62 - DNA, 435 bp, Homo sapiens
atgaaacacctgtggttcttcctcctgctggtggcagctcccagatgtgagtgtctcaaggctgcagacatggagatatgggaggtgcctctgagcccagggctcactgtgggtctctctgttcacagtggtcctgtcccaggtgcagctgcaggagtcgggcccaggactggtgaagccttcggacaccctgtccctcacctgcgctgtctctggttactccatcagcagtagtaactggtggggctggatccggcagcccccagggaagggactggagtggattgggtacatctattatagtgggagcacctactacaacccgtccctcaagagtcgagtcaccatgtcagtagacacgtccaagaaccagttctccctgaagctgagctctgtgaccgccgtggacacggccgtgtattactgtgcgagaaa

SEQ ID NO: 63 - DNA, 433 bp, Homo sapiens
atgaaacacctgtggttcttcctcctcctggtggcagctcccagatgtgagtgtctcaggaatgcggatatgaagatatgagatgctgcctctgatcccagggctcactgtgggtttctctgttcacaggggtcctgtcccaggtgcagctacagcagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatatcagtagacacgtccaagaaccagttctccctgaagctgagctctgtgaccgccgcggacacggctgtgtattactgtgcgagagg

SEQ ID NO: 64 - DNA, 439 bp, Homo sapiens
atgaagcacctgtggttcttcctcctgctggtggcggctcccagatgtgagtgtttctaggatgcagacatggagatatgggaggctgcctctgatcccagggctcactgtgggtttttctgttcacaggggtcctgtcccagctgcagctgcaggagtcgggcccaggactggtgaagccttcggagaccctgtccctcacctgcactgtctctggtggctccatcagcagtagtagttactactggggctggatccgccagcccccagggaaggggctggagtggattgggagtatctattatagtgggagcacctactacaacccgtccctcaagagtcgagtcaccatatccgtagacacgtccaagaaccagttctccctgaagctgagctctgtgaccgccgcagacacggctgtgtattactgtgcgagaca

SEQ ID NO: 65 - DNA, 432 bp, Homo sapiens
atgaaacatctgtggttcttccttctcctggtggcagctcccagatgtgagtatctcagggatccagacatggggatatgggaggtgcctctgatcccagggctcactgtgggtctctctgttcacaggggtcctgtcccaggtgcagctgcaggagtcgggcccaggactggtgaagccttcggagaccctgtccctcacctgcactgtctctggtggctccatcagtagttactactggagctggatccggcagcccccagggaagggactggagtggattgggtatatctattacagtgggagcaccaactacaacccctccctcaagagtcgagtcaccatatcagtagacacgtccaagaaccagttctccctgaagctgagctctgtgaccgctgcggacacggccgtgtattactgtgcgagaga

SEQ ID NO: 66 - DNA, 299 bp, Homo sapiens
cagctgcagctgcaggagtccggctcaggactggtgaagccttcacagaccctgtccctcacctgcgctgtctctggtggctccatcagcagtggtggttactcctggagctggatccggcagccaccagggaagggcctggagtggattgggtacatctatcatagtgggagcacctactacaacccgtccctcaagagtcgagtcaccatatcagtagacaggtccaagaaccagttctccctgaagctgagctctgtgaccgccgcggacacggccgtgtattactgtgccagaga

SEQ ID NO: 67 - DNA, 294 bp, Homo sapiens
caggtgcagctgcaggagtcgggcccaggactggtgaagccttcggagaccctgtccctcacctgcgctgtctctggttactccatcagcagtggttactactggggctggatccggcagcccccagggaaggggctggagtggattgggagtatctatcatagtgggagcacctactacaacccgtccctcaagagtcgagtcaccatatcagtagacacgtccaagaaccagttctccctgaagctgagctctgtgaccgccgcagacacggccgtgtattactgtgcgaga

SEQ ID NO: 68 - DNA, 299 bp, Homo sapiens
caggtgcagctgcaggagtcgggcccaggactggtgaagccttcacagaccctgtccctcacctgcactgtctctggtggctccatcagcagtggtgattactactggagttggatccgccagcccccagggaagggcctggagtggattgggtacatctattacagtgggagcacctactacaacccgtccctcaagagtcgagttaccatatcagtagacacgtccaagaaccagttctccctgaagctgagctctgtgactgccgcagacacggccgtgtattactgtgccagaga

SEQ ID NO: 69 - DNA, 436 bp, Homo sapiens
atggggtcaaccgccatcctcgccctcctcctggctgttctccaaggtcagtcctgccgagggcttgaggtcacagaggagaacgggtggaaaggagcccctgattcaaattttgtgtctcccccacaggagtctgtgccgaggtgcagctggtgcagtctggagcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagaca

SEQ ID NO: 70 - DNA, 306 bp, Homo sapiens
gagtctgtgccggaagtgcagctggtgcagtctggagcagaggtgaaaaagcccggggagtctctgaggatctcctgtaagggttctggatacagctttaccagctactggatcagctgggtgcgccagatgcccgggaaaggcctggagtggatggggaggattgatcctagtgactcttataccaactacagcccgtccttccaaggccacgtcaccatctcagctgacaagtccatcagcactgcctacctgcagtggagcagcctgaaggcctcggacaccgccatgtattactgtgcgaga

SEQ ID NO: 71 - DNA, 448 bp, Homo sapiens
atgtctgtctccttcctcatcttcctgcccgtgctgggcctcccatggggtcagtgtcagggagatgccgtattcacagcagcattcacagactgaggggtgtttcactttgctgtttccttttgtctccaggtgtcctgtcacaggtacagctgcagcagtcaggtccaggactggtgaagccctcgcagaccctctcactcacctgtgccatctccggggacagtgtctctagcaacagtgctgcttggaactggatcaggcagtccccatcgagaggccttgagtggctgggaaggacatactacaggtccaagtggtataatgattatgcagtatctgtgaaaagtcgaataaccatcaacccagacacatccaagaaccagttctccctgcagctgaactctgtgactcccgaggacacggctgtgtattactgtgcaagaga

SEQ ID NO: 72 - DNA, 388 bp, Homo sapiens
taaggggctccccagtcactgggctgagggagaaaccagcacagtcaagtgagacttcatgcactcccatctcctctccacaggtgcccactcccaggtgcagctggtgcaatctgggtctgagttgaagaagcctggggcctcagtgaaggtttcctgcaaggcttctggatacaccttcactagctatgctatgaattgggtgcgacaggcccctggacaagggcttgagtggatgggatggatcaacaccaacactgggaacccaacgtatgcccagggcttcacaggacggtttgtcttctccttggacacctctgtcagcacggcatatctgcagatctgcagcctaaaggctgaggacactgccgtgtattactgtgcgaga

SEQ ID NO: 73 - DNA, 149 bp, Homo sapiens
atggactggatttggagggtcctcttcttggtgggagcagcgacaggcaaggagatgccaagtcccagtgatgaggaggggattgagtccagtcaaggtggctttcatccactcctgtgttctctccacaggtgcccactcccaaatgc

SEQ ID NO: 74 - DNA, 149 bp, Homo sapiens
atggactggacctggagaatcctcttcttggtggcagcagccacaggtaaggggctcccaagtcccagtgatgaggaggggattgagtccagtcaaggtggcttttatccactcctgtgtcccctccacagatgcctactcccagatgc

SEQ ID NO: 75 - DNA, 149 bp, Homo sapiens
atggactggacctggaggatcctcttcttggtggcagcagccacaggtaagaggctccctagtcccagtgatgagaaagagattgagtccagtccagggagatctcatccacttctgtgttctctccacaggagcccactcccaggtgc

SEQ ID NO: 76 - DNA, 148 bp, Homo sapiens
atggactggacctggagcatccttttcttggtggcagcagcaacaggtaacggactccccagtcccagggctgagagagaaaccaggccagtcatgtgagacttcacccactcctgtgtcctctccacaggtgcccactcccaggttc

SEQ ID NO: 77 - DNA, 150 bp, Homo sapiens
atggactggacctggaggttcctctttgtggtggcagcagctacaggtaaggggcttcctagtcctaaggctgaggaagggatcctggtttagttaaagaggattttattcacccctgtgtcctctccacaggtgtccagtcccaggtgc

SEQ ID NO: 78 - DNA, 150 bp, Homo sapiens
atggactggacctggaggatcctcttcttggtggcagcagctacaagtaaggggcttcctagtctcaaagctgaggaacggatcctggttcagtcaaagaggattttattctctcctgtgttctctccacaggtgcccactcccaggtgc

SEQ ID NO: 79 - DNA, 6 bp, Homo sapiens
gaggtc

SEQ ID NO: 80 - DNA, 149 bp, Homo sapiens
atggactgcacctggaggatcctcttcttggtggcagcagctacaggcaagagaatcctgagttccagggctgatgaggggactgggtccagttaagtggtgtctcatccactcctctgtcctctccacaggcacccacgcccaggtcc

SEQ ID NO: 81 - DNA, 149 bp, Homo sapiens
atggactggacctggagggtcttctgcttgctggctgtagctccaggtaaagggccaactggttccagggctgaggaagggattttttccagtttagaggactgtcattctctactgtgtcctctccgcaggtgctcactcccaggtgc

SEQ ID NO: 82 - DNA, 149 bp, Homo sapiens
atggactggacctggaggatcctctttttggtggcagcagccacaggtaaggggctgccaaatcccagtgaggaggaagggatcgaagccagtcaagggggcttccatccactcctgtgtcttctctacaggtgtccactcccaggttc

SEQ ID NO: 83 - DNA, 263 bp, Homo sapiens
atggagtttgggctgagctgggttttccttgttgctattataaaaggtgatttatggagaactagagacattgagtggacgtgagtgagataagcagtgaatatatgtggcagtttctgactaggttgtctctgtgtttgcaggtgtccagtgtcaggtgcagctggtggagtctgggggaggcttggtcaagcctggagggtccctgagactctcctgtgcagcctctggattcaccttcagtgactactacatgagctgga

SEQ ID NO: 84 - DNA, 269 bp, Homo sapiens
atggagttggggctgagctgggttttccttgttgctatattagaaggtgattcatggagaactagagatattgagtgtgaatgggcatgaatgagagaaacagtgggtatgtgtggcaatttctgacttttgtgtctctgtgtttgcaggtgtccagtgtgaggtgcagctggtggagtctgggggaggcttggtacagcctggggggtccctgagactctcctgtgcagcctctggattcaccttcagtagctacgacatgcactggg

SEQ ID NO: 85 - DNA, 269 bp, Homo sapiens
atggagtttgggctgagctggattttccttgctgctattttaaaaggtgatttatggagaactagagagattaagtgtgagtggacgtgagtgagagaaacagtggatatgtgtggcagtttctgatcttagtgtctctgtgtttgcaggtgtccagtgtgaggtgcagctggtggagtctgggggaggcttggtaaagcctggggggtcccttagactctcctgtgcagcctctggattcactttcagtaacgcctggatgagctggg

SEQ ID NO: 86 - DNA, 268 bp, Homo sapiens
atggagtttgggctgagctgggttttccttgttgctattttaaaaggtgattcatggatcaatagagatgttgagtgtgagtgaacacgagtgagagaaacagtggatttgtgtggcagtttctgaccaggtgtctctgtgtttgcaggtgtccagtgtgaggtgcagctggtggagtctgggggaggtgtggtacggcctggggggtccctgagactctcctgtgcagcctctggattcacctttgatgattatggcatgagctggg

SEQ ID NO: 87 - DNA, 267 bp, Homo sapiens
atggaactggggctccgctgggttttccttgttgctattttagaaggtgaatcatggaaaagtagagagatttagtgtgtgtggatatgagtgagagaaacggtggatgtgtgtgacagtttctgaccaatgtctctctgtttgcaggtgtccagtgtgaggtgcagctggtggagtctgggggaggcctggtcaagcctggggggtccctgagactctcctgtgcagcctctggattcaccttcagtagctatagcatgaactggg

SEQ ID NO: 88 - DNA, 269 bp, Homo sapiens
atggagtttgggctgagctggctttttcttgtggctattttaaaaggtaattcatggagaaatagaaaaattgagtgtgaatggataagagtgagagaaacagtggatacgtgtggcagtttctgaccagggtttctttttgtttgcaggtgtccagtgtgaggtgcagctgttggagtctgggggaggcttggtacagcctggggggtccctgagactctcctgtgcagcctctggattcacctttagcagctatgccatgagctggg

SEQ ID NO: 89 - DNA, 267 bp, Homo sapiens
atggagtttgggctgagctgggttttcctcgttgctcttttaagaggtgattcatggagaaatagagagactgagtgtgagtgaacatgagtgagaaaaactggatttgtgtggcattttctgataacggtgtccttctgtttgcaggtgtccagtgtcaggtgcagctggtggagtctgggggaggcgtggtccagcctgggaggtccctgagactctcctgtgcagcctctggattcaccttcagtagctatggcatgcactggg

SEQ ID NO: 90 - DNA, 267 bp, Homo sapiens
atggagtttgggctgagctgggttttcctcgttgctcttttaagaggtgattcatggagaaatagagagactgagtgtgagtgaacatgagtgagaaaaactggatttgtgtggcattttctgataacggtgtccttctgtttgcaggtgtccagtgtcaggtgcagctggtggagtctgggggaggcgtggtccagcctgggaggtccctgagactctcctgtgcagcgtctggattcaccttcagtagctatggcatgcactggg

SEQ ID NO: 91 - DNA, 269 bp, Homo sapiens
atggagtttggactgagctgggttttccttgttgctattttaaaaggtgattcatggataaatagagatgttgagtgtgagtgaacatgagtgagagaaacagtggatatgtgtggcagtgtctgaccagggtgtctctgtgtttgcaggtgtccagtgtgaagtgcagctggtggagtctgggggagtcgtggtacagcctggggggtccctgagactctcctgtgcagcctctggattcacctttgatgattataccatgcactggg

SEQ ID NO: 92 - DNA, 269 bp, Homo sapiens
atggagttggggctgtgctgggttttccttgttgctattttagaaggtgattcatggaaaactagagagatttagtgtgtgtggatatgagtgagagaaacagtggatatgtgtggcagtttctgaccttggtgtctctttgtttgcaggtgtccagtgtgaggtgcagctggtggagtctgggggaggcttggtacagcctggggggtccctgagactctcctgtgcagcctctggattcaccttcagtagctatagcatgaactggg

SEQ ID NO: 93 - DNA, 269 bp, Homo sapiens
atggagtttgggcttagctgggttttccttgttgctattttaaaaggtaattcatggtgtactagagatactgagtgtgaggggacatgagtggtagaaacagtggatatgtgtggcagtttctgaccttggtgtttctgtgtttgcaggtgtccaatgtgaggtgcagctggtggagtctgggggaggcttggtacagccagggcggtccctgagactctcctgtacagcttctggattcacctttggtgattatgctatgagctggt

SEQ ID NO: 94 - DNA, 267 bp, Homo sapiens
atggagttttggctgagctgggttttccttgttgctattttaaaaggtgattcatggagaactagagatattgagtgtgagtgaacacgagtgagagaaacagtggatatgtgtggcagtttctaaccaatgtctctgtgtttgcaggtgtccagtgtgaggtgcagctggtggagtctggaggaggcttgatccagcctggggggtccctgagactctcctgtgcagcctctgggttcaccgtcagtagcaactacatgagctggg

SEQ ID NO: 95 - DNA, 269 bp, Homo sapiens
atggagtttgggctgagctgggttttccttgttgctatttttaaaggtgattcatgaggaaatagagatattgagtgtgagtggacatgagtgagagaaacagtggatttgtgtggcagtttctgaccttggtgtctctgtgtttgcaggtgtccagtgtgaggtgcagctggtggagtctggggaaggcttggtccagcctggggggtccctgagactctcctgtgcagcctctggattcaccttcagtagctatgctatgcactggg

SEQ ID NO: 96 - DNA, 269 bp, Homo sapiens
atggaattggggctgagctgggttttccttgttgctattttagaaggtgattcatggaaaactaggaagattgagtgtgtgtggatatgagtgtgagaaacagtggatttgtgtggcagtttctgaccttggtgtctctttgtttgcaggtgtccagtgtgaggtgcagctggtggagtctgggggaggcttggtccagcctggggggtccctgagactctcctgtgcagcctctggattcacctttagtagctattggatgagctggg

SEQ ID NO: 97 - DNA, 269 bp, Homo sapiens
atggagtttgggctgagctgggttttccttgttgttattttacaaggtgatttatggagaactagagatgttaagtgtgagtggacgtgagtgagagaaacagtggatttgtgtgacagtttctgaccagggtgtctctgtgtttgcaggtgtccagtgtgaggtgcagctggtggagtctgggggaggcttggtccagcctggagggtccctgagactctcctgtgcagcctctggattcaccttcagtgaccactacatggactggg

SEQ ID NO: 98 - DNA, 269 bp, Homo sapiens
atggagtttgggctgagctgggttttccttgttgctattttaaaaggtgattcatggggaactagagatactgagtgtgagtggacatgagtgagagaaacagtggacgtgtgtggcactttctgaccagggtgtctctgtgtttgcaggtgtccagtgtgaggtgcagctggtggagtccgggggaggcttggtccagcctggggggtccctgaaactctcctgtgcagcctctgggttcaccttcagtggctctgctatgcactggg

SEQ ID NO: 99 - DNA, 269 bp, Homo sapiens
atggagtttgggctgagctgggttttccttgttgctattttaaaaggtgattcatggagaactggagatatggagtgtgaatggacatgagtgagataagcagtggatgtgtgtggcagtttctgaccagggtgtctctgtgtttgcaggtgtccagtgtgaggtgcagctggtggagtccgggggaggcttagttcagcctggggggtccctgagactctcctgtgcagcctctggattcaccttcagtagctactggatgcactggg

SEQ ID NO: 100 - DNA, 257 bp, Homo sapiens
atggagttgggactgagctggattttccttttggctattttaaaaggtgattcatggagaaatagagagattgagtgtgagtggacatgagtggatttgtgtggcagtttctgaccttggtgtctctgtgtttgcaggtgtccagtgtgaagtgcagctggtggagtctgggggaggcttggtacagcctggcaggtccctgagactctcctgtgcagcctctggattcacctttgatgattatgccatgcactggg

SEQ ID NO: 101 - DNA, 109 bp, Homo sapiens
gaggtgcagctggtggagtctcggggagtcttggtacagcctggggggtccctgagactctcctgtgcagcctctggattcaccgtcagtagcaatgagatgagctggg

SEQ ID NO: 102 - DNA, 109 bp, Homo sapiens
caggtgcagctggtggagtctgggggaggcgtggtccagcctgggaggtccctgagactctcctgtgcagcctctggattcaccttcagtagctatgctatgcactggg

SEQ ID NO: 103 - DNA, 109 bp, Homo sapiens
caggtgcagctggtggagtctgggggaggcgtggtccagcctggggggtccctgagactctcctgtgcagcgtctggattcaccttcagtagctatggcatgcactggg

SEQ ID NO: 104 - DNA, 70 bp, Homo sapiens
atgaaacacctgtggttcttcctcctgctggtggcagctcccagatgtgagtgtctcaaggctgcagaca

SEQ ID NO: 105 - DNA, 70 bp, Homo sapiens
atgaaacacctgtggttcttcctcctcctggtggcagctcccagatgtgagtgtctcagggatccagaca

SEQ ID NO: 106 - DNA, 70 bp, Homo sapiens; identical to SEQ ID NO: 104

SEQ ID NO: 107 - DNA, 70 bp, Homo sapiens; identical to SEQ ID NO: 104

SEQ ID NO: 108 - DNA, 70 bp, Homo sapiens
atgaaacacctgtggttcttcctcctcctggtggcagctcccagatgtgagtgtctcaggaatgcggata

SEQ ID NO: 109 - DNA, 70 bp, Homo sapiens
atgaagcacctgtggttcttcctcctgctggtggcggctcccagatgtgagtgtttctaggatgcagaca

SEQ ID NO: 110 - DNA, 70 bp, Homo sapiens
atgaaacatctgtggttcttccttctcctggtggcagctcccagatgtgagtatctcagggatccagaca

SEQ ID NO: 111 - DNA, 140 bp, Homo sapiens
atggggtcaaccgccatcctcgccctcctcctggctgttctccaaggtcagtcctgccgagggcttgaggtcacagaggagaacgggtggaaaggagcccctgattcaaattttgtgtctcccccacaggagtctgtgcc

SEQ ID NO: 112 - DNA, 11 bp, Homo sapiens
gagtctgtgcc